(19) United States
(12) Patent Application Publication
(10) Pub. No.: US 2013/0189247 A1
(43) Pub. Date: Jul. 25, 2013

(54) Multimeric Proteins Comprising Immunoglobulin Constant Domains

(71) Applicants: David Bramhill, Tucson, AZ (US); Kurt R. Gehlsen, Tucson, AZ (US); Dimiter S. Dimitrov, Frederick, MD (US); Rui Gong, Frederick, MD (US)

(72) Inventors: David Bramhill, Tucson, AZ (US); Kurt R. Gehlsen, Tucson, AZ (US); Dimiter S. Dimitrov, Frederick, MD (US); Rui Gong, Frederick, MD (US)

(73) Assignee: RESEARCH CORPORATION TECHNOLOGIES, INC., Tucson, AZ (US)

(21) Appl. No.: 13/578,538

(22) PCT Filed: Feb. 11, 2011
(86) PCT No.: PCT/US11/24552

§ 371 Date: Sep. 24, 2012

Related U.S. Application Data
(60) Provisional application No. 61/304,302, filed on Feb. 12, 2010.

(51) Int. Cl. C07K 16/00

(52) U.S. Cl. CPC C07K 16/00
USPC 424/133.1; 530/387.3; 436/501; 424/178.1

(57) ABSTRACT

The present invention relate to small binding proteins comprising two or more protein domains derived from a CH2 domain or CH2-like domain of an immunoglobulin in which the CH2 domains have been altered to recognize one or more target proteins and, in some embodiments, retain, or have modified, certain secondary effector functions.

### CROSS-REFERENCE TO RELATED APPLICATIONS

The present application is a non-provisional application claiming priority to U.S. Provisional Patent Application Ser. No. 61/304,302, filed Feb. 12, 2010, the disclosure of which is incorporated in its entirety herein by reference.

This invention was made with government support under CRADA 02461-08 awarded by the National Institute of Health. The government has certain rights in the invention.

### FIELD OF THE INVENTION

The present invention is directed to the field of immunology, particularly to small binding proteins comprising two or more protein domains derived from a CH2 domain or CH2-like domain of an immunoglobulin in which the CH2 domains have been altered to recognize one or more target proteins and, in some embodiments, retain, or have modified, certain secondary effector functions.

### BACKGROUND OF THE INVENTION

Immunoglobulins (antibodies) in adult humans are categorized into five different isotypes: IgA, IgD, IgE, IgG, and IgM. The isotypes vary in size and sequence. On average, each immunoglobulin has a molecular weight of about 150 kDa. It is well known that each immunoglobulin comprises two heavy chains (H) and two light chains (L), which are arranged to form a Y-shaped molecule. The Y-shape can be conceptually divided into the F_{ab} region, which represents the top portion of the Y-shaped molecule, and the F_{c} region, which represents the bottom portion of the Y-shaped molecule.

The heavy chains in IgG, IgA, and IgD each have a variable domain (VH) at one end followed by three constant domains: CH1, CH2, and CH3. The CH1 and CH2 regions are joined by a distinct hinge region. A CH2 domain may or may not include the hinge region. The heavy chains in IgM and IgE each have a variable domain (VH) at one end followed by four constant domains: CH1, CH2, CH3, and CH4. Sequences of the variable domains vary, but the constant domains are generally conserved among all antibodies in the same isotype.

The F_{ab} region of immunoglobulins contains the variable (V) domain and the CH1 domain; the F_{c} region of immunoglobulins contains the hinge region and the remaining constant domains, either CH2 and CH3 in IgG, IgA, and IgD, or CH2, CH3, and CH4 in IgM and IgE.

Target antigen specificity of the immunoglobulins is conferred by the paratope in the F_{ab} region. Effector functions (e.g., complement activation, interaction with F_{c} receptors such as pro-inflammatory F_{c}γ receptors, binding to various immune cell such as phagocytes, lymphocytes, platelets, mast cells, and the like) of the immunoglobulins are conferred by the F_{c} region. The F_{c} region is also important for maintaining serum half-life. Serum half-life of an immunoglobulin is mediated by the binding of the F_{c} region to the neonatal receptor FcRn. The alpha domain is the portion of FcRn that interacts with the CH2 domain (and possibly CH3 domain) of IgG, and possibly IgA, and IgD or with the CH3 domain (and possibly CH4 domain) of IgM and IgE.

Examining the constant domains of the immunoglobulin heavy chains more closely, the CH3 domains of IgM and IgE are closely related to the CH2 domain in terms of sequence and function. Without wishing to limit the present invention to any theory or mechanism, it is believed that the CH2 domain (or the equivalent CH3 domain of IgM or IgE) is responsible for all or most of the interaction with F_{c} receptors (e.g., F_{c}γ receptors), and contains histidine (His) residues important for serum half-life maintenance. The CH2 domain (or the equivalent CH3 domain of IgM or IgE) also has binding sites for complement. The CH2/CH3 domain's retention of functional characteristics of the antibody from which it is derived (e.g., interaction with F_{c}γ receptors, binding sites for complement, solubility, stability/half-life, etc.) is discussed in Dimitrov (2009) mAbs 1:1-3 and Dimitrov (2009) mAbs 1:26-28. Prabakaran et al. (2008, Acta Crystallogr D Biol Crystallogr 64:1062-1067) compared the structure of a CH2 IgG domain lacking N-linked glycosylation at Asn297 to the structure of a wild type CH2 IgG domain and found the two CH2 domains to have extremely similar structures. Without wishing to limit the present invention to any theory or mechanisms, it is believed that some modifications to the CH2 domain may have only small effects on the overall structure of the CH2 domain (or CH2-like domain), and it is likely that in cases where the modified CH2 structure was similar to the wild-type CH2 structure the modified CH2 domain would confer the same functional characteristics as the wild-type CH2 domain possessed in the full immunoglobulin molecule.

The present invention features multimeric CH2 domains (CH2Ds) comprising two or more CH2Ds, in some cases being linked via a linker. The multimeric CH2Ds of the present invention can effectively bind a single or multiple target antigens. In some examples, the multimeric CH2Ds may be engineered to have multiple specificities to Fc receptors (each monomer could bind to distinct Fc receptors to target a specific effector function), or limited to only one functional binding site for pro-inflammatory F_{c} receptors (e.g., F_{c}γ receptors) and/or substantially lack complement activation capabilities. These features may be important for regulating effector functions (e.g., binding to various immune cell such as phagocytes, lymphocytes, platelets, mast cells, and the like), for example helping to prevent adverse immune effects, or in another example, to enhance the immune response to treat a disease. In some embodiments, the multimeric CH2Ds of the present invention have an increased serum half-life as compared to the individual monomers. Increased serum half-life may be conferred via additional binding sites for FcRn, or via modified binding sites for FcRn having more effective interactions with FcRn, or by virtue of having multiple CH2Ds linked with inherent FcRn interactions.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description.

### SUMMARY

The present invention features multimeric CH2Ds. For example, the present invention features a CH2 multimer assembly comprising at least a first immunoglobulin CH2 domain linked to a second immunoglobulin CH2 domain. In some embodiments, the multimeric CH2Ds comprises no more than one CH2D that retains a functional binding site able to activate pro-inflammatory FcγR; a second CH2D containing no more than one site able to bind complement; and/or at least two functional FcRn binding sites, wherein the FcRn binding sites are wild type or modified. In another embodiment, each CH2D of a multimer retains all Fc effector functions, alternatively, each CH2D in a multimer is devoid of any Fc-effector functions.

The first immunoglobulin CH2 domain and/or the second immunoglobulin CH2 domain (or additional CH2 domains) may comprise a CH2 domain of an IgG, IgA, or IgD, a CH3 domain of an IgE or IgM, or a fragment thereof. In some embodiments, the multimer CH2D comprises at least one CH2 domain connected via a linker to one or more of the following: other CH2D, an immunoglobulin CH1 domain, an IgG CH3 domain, an entire immunoglobulin VH domain, and/or an entire immunoglobulin VL domain, or any combination thereof.

The first immunoglobulin CH2D and a second immunoglobulin CH2D (or other immunoglobulin domains) may be linked via a linker of various lengths, for example between 5 to 20 amino acids. The linker may comprise at least one multimerizing domain. The linker may comprise a hinge region or fragment of a hinge region.

The multimeric CH2Ds may be arranged in various configurations. For example, the N-terminus of the first immunoglobulin CH2D may be linked to the C-terminus of the second immunoglobulin CH2D, the N-terminus of the second immunoglobulin CH2D may be linked to the C-terminus of the first immunoglobulin CH2D, the C-terminus of the first immunoglobulin CH2D may be linked to a C-terminus of the second immunoglobulin CH2D, the N-terminus of the first immunoglobulin CH2D may be linked to an N-terminus of the second immunoglobulin CH2 domain, forming a variety of dimers, trimers and tetramers. All or some of the multimeric CH2Ds may be stabilized CH2Ds.

The multimer CH2D may comprise at least one complementary-determining region (CDR) loop or a functional fragment thereof from an immunoglobulin molecule. For example, one or more loops of either the first or second (or both) CH2Ds may be entirely or partially replaced with one or more CDRs or functional fragments thereof. In some embodiments, at least one loop of the first or second (or both) CH2Ds is modified. In some embodiments, at least one strand of the first or second CH2D (or both) is modified. In some embodiments, at least one loop and at least one strand of the first or second CH2D (or both) are modified.

In some embodiments, only one immunoglobulin CH2D has a functional Fc receptor-binding region for binding to a target Fc receptor to effectively activate an immune response. In some embodiments, at least one immunoglobulin CH2D does not have a functional Fc receptor-binding region for binding to a target Fc receptor to effectively activate an immune response. In another embodiment, all the CH2Ds in a multimer retain Fc receptor-binding.

The multimer CH2D may have a greater serum half-life as compared to that of either the first CH2D immunoglobulin domain alone or the second CH2 immunoglobulin domain alone. In some embodiments, the multimer CH2D comprises at least one or at least two functional FcRn binding sites (e.g., modified, wild-type, etc.). In some embodiments, the multimer CH2D comprises no more than one binding site for binding to complement. In some embodiments, at least one immunoglobulin CH2D is modified so as to reduce or eliminate complement activation. In some embodiments, at least one immunoglobulin CH2D is derived from an immunoglobulin isotype having reduced or absent activation of complement.

The CH2D may have a greater avidity in binding a target as compared to that of either the first CH2D2 immunoglobulin domain alone or the second CH2 immunoglobulin domain alone.

The multimer CH2D may be specific for one or more targets. For example, both the first and second CH2Ds are specific for a first target. In some embodiments, the first CH2D is specific for a first target and the second CH2D is specific for a second target. If the CH2D comprises more than two CH2Ds, the additional CH2Ds may be specific for a target for which the first immunoglobulin CH2D is specific, a target for which the second immunoglobulin CH2D is specific, a target for which both the first and second CH2Ds are specific, or a target for which neither the first and second CH2 domains are specific.

The CH2D may also be modified to selectively target one or more Fc receptors. For example, the CH2D from IgE could be modified to only bind the Fc-epsilon receptor and act as an antagonist. In another example, the CH2D could be modified to only bind the Fc-gamma III receptor on NK cells. Multimers could be modified to bind the same or different Fc receptors to initiate a variety of immune responses.

The present invention also features methods of treating or managing a disease or a condition of a mammal. Briefly, the method may comprise obtaining a CH2D multimer comprising at least a first immunoglobulin CH2D to a disease specific target and a second immunoglobulin CH2 domain to the same or a complementary target; introducing the CH2D multimer into a tissue of the mammal; the CH2D binds to a first target, the second CH2D binds to another epitope on the first target or binds to a second target, the binding to the target, or the recruitment of secondary Fc-effector functions, cause neutralization or destruction of the first target or targeted disease cell. In some embodiments, the CH2D monomer or multimer comprises an agent (e.g., chemical, peptide, toxin, etc.) linked to a CH2D, wherein the agent functions to neutralize or destroy the target. The agent may be inert or have reduced activity when it is linked to the CH2D. The agent may be activated or released upon uptake or recycling in a cell, or by enzymatic activation in a tissue of interest.

The present invention also features methods of detecting a disease or a condition in a mammal. Briefly, the method may comprise obtaining a CH2D multimer comprising a first immunoglobulin CH2D linked to a second immunoglobulin CH2D; introducing the CH2D multimer into a sample of the mammal; detecting binding of the CH2D multimer to a target in the sample, the target being associated with the disease or condition, wherein detecting the binding of the polypeptide to the target in the sample is indicative of the disease or condition. The CH2D multimer may be linked to a number of imaging or detecting agents, including, but not limited to: fluorescent compounds, radioactive compounds, compounds for PET, MRI, CT or X-ray imaging, or be tagged with a molecule that allows for detection by another CH2D or method.

The present invention also features methods of identifying a CH2D multimer that specifically binds a target. Briefly, the method may comprise providing a library of particles displaying on their surface a CH2D comprising at least a first immunoglobulin CH2D linked to a second immunoglobulin CH2D; introducing the target to the library of particles; and selecting particles from the library that specifically bind to the target. In some embodiments, CH2D monomers may be displayed on the library particles and the selected monomers joined by linkers.

The present invention also features pharmaceutical compositions. For example, the pharmaceutical compositions may comprise a CH2D multimer comprising a first immunoglobulin CH2D linked to at least a second immunoglobulin CH2D. The pharmaceutical compositions may comprise a CH2D multimer comprising at least a first immunoglobulin CH2D linked to a second immunoglobulin CH2D, wherein the CH2 multimer comprises at least one functional binding site able to activate Fc receptors; at least one site able to bind complement; and at least one functional FcRn binding sites, wherein the FcRn binding sites are wild type or modified.

In some embodiments, the pharmaceutical compositions comprise monomers, for example a first immunoglobulin CH2D. In some embodiments, the CH2D comprises at least one functional binding site able to activate a variety of Fc receptors; at least one site able to bind complement; and at least one functional FcRn binding sites, wherein the FcRn binding sites are wild type or modified. The monomers may be stabilized CH2 domains.

In some embodiments, the pharmaceutical compositions comprise a polypeptide comprising a first immunoglobulin CH2D, wherein the CH2D comprises an N-terminal truncation of about 1 to about 7 amino acids, and wherein (i) at least one loop of the CH2D is mutated; (ii) at least a portion of a loop region of the CH2D is replaced by a complementarity determining region (CDR), or a functional fragment thereof, from an immunoglobulin variable domain; or (iii) both, wherein the first immunoglobulin CH2D specifically binds an antigen. The first immunoglobulin CH2D may have a molecular weight of less than about 15 kDa, however the first immunoglobulin domain is not limited to this size.

### DEFINITIONS

In order to facilitate the review of the various embodiments of the invention, the following explanations of specific terms are provided:

Definitions of common terms in molecular biology, cell biology, and immunology may be found in Kuby Immunology, Thomas J. Kindt, Richard A. Goldsby, Barbara Anne Osborne, Janis Kuby, published by W.H. Freeman, 2007 (ISBN 1429202114); and Genes IX, Benjamin Lewin, published by Jones & Bartlett Publishers, 2007 (ISBN-10: 0763740632).

Antibody:

A protein (or complex) that includes one or more polypeptides substantially encoded by immunoglobulin genes or fragments of immunoglobulin genes. The immunoglobulin genes may include the kappa, lambda, alpha, gamma, delta, epsilon, and mu constant region genes, as well as the myriad of immunoglobulin variable region genes. Light chains may be classified as either kappa or lambda. Heavy chains may be classified as gamma, mu, alpha, delta, or epsilon, which in turn define the immunoglobulin classes IgG, IgM, IgA, IgD, and IgE, respectively.

As used herein, the term “antibodies” includes intact immunoglobulins as well as fragments (e.g., having a molecular weight between about 10 kDa to 100 kDa). Antibody fragments may include: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody pepsin, followed by reduction, to yield an intact light chain and a portion of the heavy chain; two Fab′ fragments are obtained per antibody molecule; (3) (Fab′)2, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; (6) scFv, single chain antibody, a genetically engineered molecule containing the variable region of the light chain, the variable region of the heavy chain, linked by a suitable polypeptide linker as a genetically fused single chain molecule; and (7) CH1 domains, CH2 domains, CH3 domains, CH4 domains, and the like. Methods of making antibody fragments are routine (see, for example, Harlow and Lane, Using Antibodies: A Laboratory Manual, CSHL, New York, 1999).

Antibodies can be monoclonal or polyclonal. Merely by way of example, monoclonal antibodies can be prepared from murine hybridomas according to classical methods such as Kohler and Milstein (Nature 256:495-97, 1975) or derivative methods thereof. Detailed procedures for monoclonal antibody production are described, for example, by Harlow and Lane, Using Antibodies: A Laboratory Manual, CSHL, New York, 1999.

A “humanized” immunoglobulin, such as a humanized antibody, is an immunoglobulin including a human framework region and one or more CDRs from a non-human (e.g., mouse, rat, synthetic, etc.) immunoglobulin. The non-human immunoglobulin providing the CDR is termed a “donor,” and the human immunoglobulin providing the framework is termed an “acceptor.” A humanized antibody binds to the same or similar antigen as the donor antibody that provides the CDRs. The molecules can be constructed by means of genetic engineering (see, for example, U.S. Pat. No. 5,585,089).

Antigen:

A compound, composition, or substance that can stimulate the production of antibodies or a T-cell response, including compositions that are injected or absorbed. An antigen reacts with the products of specific humoral or cellular immunity. In some embodiments, an antigen also may be the specific binding target of the CH2Ds whether or not such interaction could produce an immunological response.

Avidity:

binding affinity (e.g., increased) as a result from bivalent or multivalent binding sites that may simultaneously bind to a multivalent target antigen or receptor that is either itself multimeric or is present on the surface of a cell or virus such that it is able to be organized into a multimeric form. For example, the two Fab arms of an immunoglobulin can provide such avidity increase for an antigen compared with the binding of a single Fab arm, since both sites must be unbound for the immunoglobulin to dissociate.

Binding Affinity:

The strength of binding between a binding site and a ligand (e.g., between an antibody, a CH2 domain, or a CH3 domain and an antigen or epitope). The affinity of a binding site X for a ligand Y is represented by the dissociation constant (Kd), which is the concentration of Y that is required to occupy half of the binding sites of X present in a solution. A lower (Kd) indicates a stronger or higher-affinity interaction between X and Y and a lower concentration of ligand is needed to occupy the sites. In general, binding affinity can be affected by the alteration, modification and/or substitution of one or more amino acids in the epitope recognized by the paratope (portion of the molecule that recognizes the epitope). Binding affinity can also be affected by the alteration, modification and/or substitution of one or more amino acids in the paratope. Binding affinity can be the affinity of antibody binding an antigen.

In one example, binding affinity is measured by end-point titration in an Ag-ELISA assay. Binding affinity is substantially lowered (or measurably reduced) by the modification and/or substitution of one or more amino acids in the epitope recognized by the antibody paratope if the end-point titer of a specific antibody for the modified/substituted epitope differs by at least 4-fold, such as at least 10-fold, at least 100-fold or greater, as compared to the unaltered epitope.

CH2 or CH3 Domain Molecule:

A polypeptide (or nucleic acid encoding a polypeptide) derived from an immunoglobulin CH2 or CH3 domain. The immunoglobulin can be IgG, IgA, IgD, IgE or IgM. The CH2 or CH3 molecule is composed of a number of parallel β-strands connected by loops of unstructured amino acid sequence. In one embodiment described herein, the CH2 or CH3 domain molecule comprises at least one CDR, or functional fragment thereof. The CH2 or CH3 domain molecule can further comprise additional amino acid sequence, such as a complete hypervariable loop. In another embodiment, the CH2 or CH3 domain molecules have at least a portion of one or more loop regions replaced with a CDR, or functional fragment thereof. In some embodiments described herein, the CH2 or CH3 domains comprise one or more mutations in a loop region of the molecule. A “loop region” of a CH2 or CH3 domain refers to the portion of the protein located between regions of β-sheet (for example, each CH2 domain comprises seven β-sheets, A to G, oriented from the N- to C-terminus). A CH2 domain comprises six loop regions: Loop 1, Loop 2, Loop 3, Loop A-B, Loop C-D and Loop E-F. Loops A-B, C-D and E-F are located between β-sheets A and B, C and D, and E and F, respectively. Loops 1, 2 and 3 are located between β-sheets B and C, D and E, and F and G, respectively. The CH2 and CH3 domain molecules disclosed herein can also comprise an N-terminal deletion, such as a deletion of about 1 to about 7 amino acids. In particular examples, the N-terminal deletion is 1, 2, 3, 4, 5, 6 or 7 amino acids in length. The CH2 and CH3 domain molecules disclosed herein can also comprise a C-terminal deletion, such as a deletion of about 1 to about 4 amino acids. In particular examples, the C-terminal deletion is 1, 2, 3 or 4 amino acids in length.

CH2 and CH3 domain molecules are small in size, usually less than 15 kDa. The CH2 and CH3 domain molecules can vary in size depending on the length of CDR/hypervariable amino acid sequence inserted in the loops regions, how many CDRs are inserted and whether another molecule (such as an effector molecule or label) is conjugated to the CH2 or CH3 domain. In some embodiments, the CH2 or CH3 domain molecules do not comprise additional constant domains (e.g. CH1 or another CH2 or CH3 domain) or variable domains. In one embodiment, the CH2 domain is from IgG, IgA or IgD. In another embodiment, the constant domain is a CH3 domain from IgE or IgM, which is homologous to the CH2 domains of IgG, IgA or IgD.

The CH2 and CH3 domain molecules provided herein can be glycosylated or unglycosylated. For example, a recombinant CH2 or CH3 domain can be expressed in an appropriate yeast, insect, plant or mammalian cell to allow glycosylation of the molecule at one or more natural or engineered glycosylation sites in the protein. The recombinant CH2 or CH3 domains can be expressed with a mixture of glycosylation patterns as typically results from the production in a mammalian cell line like CHO (Schroder et al., Glycobiol 20(2):248-259, 2010; Hossler et al., Glycobiol 19(9):936-949, 2009) or the CH2 domains can be made with substantially homogeneous (greater than 50% of one type) glycopatterns. A method of homogenously or nearly homogenously glycosylating recombinant proteins has been developed in genetically-engineered yeast (Jacobs et al., Nature Protocols 1(4):58-70, 2009). The glycans added to the protein may be the same as occur naturally or may be forms not usually found on human glycoproteins. Non-limiting examples include Man5, GnMan5, GalGnMan5 GnMan3, GalGnMan3, Gn2Man3, Gal2Gn2Man3. In vitro reactions may be used to add additional components (such as sialic acid) to the glycans added in the recombinant production of the glycoprotein. Addition of different glycans may provide for improvements in half-life, stability, and other pharmaceutical properties. As is well known the presence of fucose in the usual N-glycans of the CH2 domain of antibodies affects ADCC (antibody dependent).

The CH2 and CH3 domain molecules provided herein can be stabilized or native molecules. Stabilized CH2Ds have certain alterations in their amino acid sequence to allow additional disulfide bonds to be formed without noticeable alteration of the protein's functions (WO 2009/099961A2).

Complementarity Determining Region (CDR):

A short amino acid sequence found in the variable domains of antigen receptor (such as immunoglobulin and T cell receptor) proteins that provides the receptor with contact sites for antigen and its specificity for a particular antigen. Each polypeptide chain of an antigen receptor contains three CDRs (CDR1, CDR2 and CDR3). Antigen receptors are typically composed of two polypeptide chains (a heavy chain and a light chain), therefore there are six CDRs for each antigen receptor that can come into contact with the antigen. Since most sequence variation associated with antigen receptors are found in the CDRs, these regions are sometimes referred to as hypervariable domains.

CDRs are found within loop regions of an antigen receptor (usually between regions of β-sheet structure). These loop regions are typically referred to as hypervariable loops. Each antigen receptor comprises six hypervariable loops: H1, H2, H3, L1, L2 and L3. For example, the H1 loop comprises CDR1 of the heavy chain and the L3 loop comprises CDR3 of the light chain. The CH2 and CH3 domain molecules described herein may comprise engrafted amino acids sequences from a variable domain of an antibody. The engrafted amino acids comprise at least a portion of a CDR. The engrafted amino acids can also include additional amino acid sequence, such as a complete hypervariable loop. As used herein, a “functional fragment” of a CDR is at least a portion of a CDR that retains the capacity to bind a specific antigen. The loops may be mutated or rationally designed.

A numbering convention for the location of CDRs is described by Kabat et al. 1991, Sequences of Proteins of Immunological Interest, 5^{th} Edition, U.S. Department of Health and Human Services, Public Health Service, National Institutes of Health, Bethesda, Md. (NIH Publication No. 91-3242).

Contacting:

Placement in direct physical association, which includes both in solid and in liquid form.

Degenerate Variant:

As used herein, a “degenerate variant” of a CH2 or CH3 domain molecule is a polynucleotide encoding a CH2 or CH3 domain molecule that includes a sequence that is degenerate as a result of redundancies in the genetic code. There are 20 natural amino acids, most of which are specified by more than one codon. Therefore, all degenerate nucleotide sequences are included as long as the amino acid sequence of the CH2 or CH3 domain molecule encoded by the nucleotide sequence is unchanged.

The use of degenerate variant sequences that encode the same polypeptide is of great utility in the expression of recombinant multimeric forms of CH2Ds. Linear gene constructs that use extensive repeats of the same DNA sequence are prone to deletion due to recombination. This can be minimized by the selection of codons that encode the same amino acids yet differ in sequence, designing the gene to avoid repeated DNA elements even though it encodes a repeated amino acid sequence, such as a linear dimer CH2D comprising two identical CH2Ds. Even if a dimer has different CH2Ds, much or all of the scaffold amino acid sequence may be identical, and certain trimeric CH2Ds may have identical linkers. Similar codon selection principles can be used to reduce repeats in a gene encoding any linear repeated domains, such as variable heavy chain multimers, Fibronectin domain multimers, ankyrin repeat proteins or other scaffold multimers.

Domain:

A protein structure which retains its tertiary structure independently of the remainder of the protein. In some cases, domains have discrete functional properties and can be added, removed or transferred to another protein without a loss of function.

Effector Molecule:

A molecule, or the portion of a chimeric molecule, that is intended to have a desired effect on a cell to which the molecule or chimeric molecule is targeted. An effector molecule is also known as an effector moiety (EM), therapeutic agent, or diagnostic agent, or similar terms.

Therapeutic Agents

include such compounds as nucleic acids, proteins, peptides, amino acids or derivatives, glycoproteins, radioisotopes, lipids, carbohydrates, or recombinant viruses. Nucleic acid therapeutic and diagnostic moieties include antisense nucleic acids, derivatized oligonucleotides for covalent cross-linking with single or duplex DNA, and triplex forming oligonucleotides. Alternatively, the molecule linked to a targeting moiety, such as a CH2 or CH3 domain molecule, may be an encapsulation system, such as a liposome or micelle that contains a therapeutic composition such as a drug, a nucleic acid (such as an antisense nucleic acid), or another therapeutic moiety that can be shielded from direct exposure to the circulatory system. Means of preparing liposomes attached to antibodies are well known to those of skill in the art. See, for example, U.S. Pat. No. 4,957,735; and Connor et al. 1985, Pharm. Ther. 28:341-365. Diagnostic agents or moieties include radioisotopes and other detectable labels. Detectable labels useful for such purposes are also well known in the art, and include radioactive isotopes such as ³²P, ¹²⁵I, and ¹³¹I, fluorophores, chemiluminescent agents, and enzymes.

Epitope:

An antigenic determinant. These are particular chemical groups or contiguous or non-contiguous peptide sequences on a molecule that are antigenic, that is, that elicit a specific immune response. An antibody binds a particular antigenic epitope based on the three dimensional structure of the antibody and the matching (or cognate) epitope.

Expression:

The translation of a nucleic acid into a protein. Proteins may be expressed and remain intracellular, become a component of the cell surface membrane, or be secreted into the extracellular matrix or medium

Expression Control Sequences:

Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, a start codon (i.e., ATG) in front of a protein-encoding gene, splicing signal for introns, and maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

A promoter is an array of nucleic acid control sequences that directs transcription of a nucleic acid. A promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Both constitutive and inducible promoters are included (see, for example, Bitter et al. (1987) Methods in Enzymology 153:516-544).

Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see, for example, Bitter et al. (1987) Methods in Enzymology 153:516-544). For example, when cloning in bacterial systems, inducible promoters such as pL of bacteriophage lambda, plac, ptrp, ptac (ptrp-lac hybrid promoter) and the like may be used. In some embodiments, when cloning in mammalian cell systems, promoters derived from the genome of mammalian cells (such as the metallothionein promoter) or from mammalian viruses (such as the retrovirus long terminal repeat; the adenovirus late promoter; the vaccinia virus 7.5 K promoter, etc.) can be used. Promoters produced by recombinant DNA or synthetic techniques may also be used to provide for transcription of the nucleic acid sequences.

A polynucleotide can be inserted into an expression vector that contains a promoter sequence that facilitates the efficient transcription of the inserted genetic sequence of the host. The expression vector typically contains an origin of replication, a promoter, as well as specific nucleic acid sequences that allow phenotypic selection of the transformed cells.

Fc Binding Regions.

The FcRn binding region of the CH2D is known to comprise the amino acid residues M252, I253, S254, T256, V259, V308, H310, Q311 (Kabat numbering of IgG). These amino acid residues have been identified from studies of the full IgG molecule and/or the Fc fragment to locate the residues of the CH2 domain that directly affect the interaction with FcRn. Three lines of investigation have been particularly illuminating: (a) crystallographic studies of the complexes of FcRn bound to Fc, (b) comparisons of the various human isotypes (IgG1, IgG2, IgG3 and IgG4) with each other and with IgGs from other species that exhibit differences in FcRn binding and serum half-life, correlating the variation in properties to specific amino acid residue differences, and (c) mutation analysis, particularly the isolation of mutations that show enhanced binding to FcRn, yet retain the pH-dependence of FcRn interaction. All three approaches highlight the same regions of CH2D as crucial to the interaction with FcRn. The CH3 domain of IgG also contributes to the interaction with FcRn, but the protonation/deprotonation of H310 is thought to be primarily responsible and sufficient for the pH dependence of the interaction.

Fc Receptor and Complement Binding Regions of CH2D

Apart from FcRn, the CH2 domain is involved in binding other Fc receptors and also complement. The region of the CH2D involved in these interactions comprises the amino acid residues E233, L234, L235, G236, G237, P238, Y296, N297, E318, K320, K322, N327, (Kabat numbering of IgG). These amino acid residues have been identified from studies of the full IgG molecule and/or the Fc fragment to locate the residues of the CH2 domain that directly affect the interaction with Fc receptors and with complement. Three lines of investigation have been useful: (a) crystallographic studies of the complexes of a receptor (e.g. Fc□RIIIa) bound to Fc, (b) sequence comparisons of the various human IgG isotypes (IgG1, IgG2, IgG3 and IgG4) and other immunoglobulin classes that exhibit differences in Fc Receptor binding, binding to complement or induction of pro-inflammatory or anti-inflammatory signals, correlating the variation in properties to specific amino acid residue differences, and (c) the isolation of mutations that show reduced or enhanced binding to Fc receptors or complement. The CH3 domain of IgG may contribute to the interaction with some Fc receptors (e.g. Fc□RIa); however, the CH1-proximal end of the CH2 in the IgG molecule is the primary region of interaction, and the mutations in the CH3 domain of IgG may enhance Fc interaction with Fc□RIa indirectly, perhaps by altering the orientation or the accessibility of certain residues of the CH2 domain. Additionally, though the residues are very close to the Fc□RIIIa interaction site of CH2 revealed in the crystal structure, N297 may affect binding because it is the site of N-linked glycosylation of the CH2 domain. The state and nature of the N-linked glycan affect binding to Fc receptors (apart from FcRn); for example, glycosylated IgG binds better than unglycosylated IgG, especially when the glycoform lacks fucose. Greenwood J, Clark M, Waldmann H. Structural motifs involved in human IgG antibody effector functions Eur J Immunol 1993; 5: 1098-1104

Framework Region:

Amino acid sequences interposed between CDRs (or hypervariable regions). Framework regions include variable light and variable heavy framework regions. Each variable domain comprises four framework regions, often referred to as FR1, FR2, FR3 and FR4. The framework regions serve to hold the CDRs in an appropriate orientation for antigen binding. Framework regions typically form β-sheet structures.

Fungal-Associated Antigen (FAAs):

A fungal antigen that can stimulate fungal-specific T-cell-defined immune responses. Exemplary FAAs include, but are not limited to, an antigen from Candida albicans, Cryptococcus (such as d25, or the MP98 or MP88 mannoprotein from C. neoformans, or an immunological fragment thereof), Blastomyces (such as B. dermatitidis, for example WI-I or an immunological fragment thereof), and Histoplasma (such as H. capsulatum).

Heterologous:

A heterologous polypeptide or polynucleotide refers to a polypeptide or polynucleotide derived from a different source or species.

Hypervariable Region:

Regions of particularly high sequence variability within an antibody variable domain. The hypervariable regions form loop structures between the β-sheets of the framework regions. Thus, hypervariable regions are also referred to as “hypervariable loops.” Each variable domain comprises three hypervariable regions, often referred to as H1, H2 and H3 in the heavy chain, and L1, L2 and L3 in the light chain.

Immune Response:

A response of a cell of the immune system, such as a B-cell, T-cell, macrophage or polymorphonucleocyte, to a stimulus such as an antigen. An immune response can include any cell of the body involved in a host defense response for example, an epithelial cell that secretes an interferon or a cytokine. An immune response includes, but is not limited to, an innate immune response or inflammation.

Immunoconjugate:

A covalent linkage of an effector molecule to an antibody or a CH2 or CH3 domain molecule. The effector molecule can be a detectable label, biologically active protein, drug, toxin or an immunotoxin. Specific, non-limiting examples of immunotoxins include, but are not limited to, abrin, ricin, Pseudomonas exotoxin (PE, such as PE35, PE37, PE38, and PE40), diphtheria toxin (DT), botulinum toxin, or modified toxins thereof. Other toxins that may be attached to an antibody or CH2 or CH3 domain include auristatin, maytansinoids, and cytolytic peptides. Other immunoconjugates may be composed of antibodies or CH2 or CH3 domains linked to drug molecules (ADC or “antibody drug conjugates”; Ducry and Stump, Bioconj Chem 21: 5-13, 2010; Erikson et al., Bioconj Chem 21: 84-92, 2010). These immunotoxins may directly or indirectly inhibit cell growth or kill cells. For example, PE and DT are highly toxic compounds that typically bring about death through liver toxicity. PE and DT, however, can be modified into a form for use as an immunotoxin by removing the native targeting component of the toxin (such as domain Ia of PE and the B chain of DT) and replacing it with a different targeting moiety, such as a CH2 or CH3 domain molecule. In one embodiment, a CH2 or CH3 domain molecule is joined to an effector molecule (EM). ADCs delivery therapeutic molecules to their conjugate binding partners. The effector molecule may be a small molecule drug or biologically active protein, such as erythropoietin. In another embodiment the effector molecule may be another immunoglobulin domain, such as a VH or CH1 domain. In another embodiment, a CH2 or CH3 domain molecule joined to an effector molecule is further joined to a lipid or other molecule to a protein or peptide to increase its half-life in the body. The linkage can be either by chemical or recombinant means. “Chemical means” refers to a reaction between the CH2 or CH3 domain molecule and the effector molecule such that there is a covalent bond formed between the two molecules to form one molecule. A peptide linker (short peptide sequence) can optionally be included between the CH2 or CH3 domain molecule and the effector molecule. Such a linker may be subject to proteolysis by an endogenous or exogenous linker to release the effector molecule at a desired site of action. Because immunoconjugates were originally prepared from two molecules with separate functionalities, such as an antibody and an effector molecule, they are also sometimes referred to as “chimeric molecules.” The term “chimeric molecule,” as used herein, therefore refers to a targeting moiety, such as a ligand, antibody or CH2 or CH3 domain molecule, conjugated (coupled) to an effector molecule.

The terms “conjugating,” “joining,” “bonding” or “linking” refer to making two polypeptides into one contiguous polypeptide molecule, or to covalently attaching a radionucleotide or other molecule to a polypeptide, such as a CH2 or CH3 domain molecule. In the specific context, the terms can in some embodiments refer to joining a ligand, such as an antibody moiety, to an effector molecule (“EM”).

Immunogen:

A compound, composition, or substance which is capable, under appropriate conditions, of stimulating an immune response, such as the production of antibodies or a T-cell response in an animal, including compositions that are injected or absorbed into an animal.

Isolated:

An “isolated” biological component (such as a nucleic acid molecule or protein) that has been substantially separated or purified away from other biological components from which the component naturally occurs (for example, other biological components of a cell), such as other chromosomal and extra-chromosomal DNA and RNA and proteins, including other antibodies. Nucleic acids and proteins that have been “isolated” include nucleic acids and proteins purified by standard purification methods. An “isolated antibody” is an antibody that has been substantially separated or purified away from other proteins or biological components such that its antigen specificity is maintained. The term also embraces nucleic acids and proteins (including CH2 and CH3 domain molecules) prepared by recombinant expression in a host cell, as well as chemically synthesized nucleic acids or proteins, or fragments thereof.

Label:

A detectable compound or composition that is conjugated directly or indirectly to another molecule, such as an antibody or CH2 or CH3 domain molecule, to facilitate detection of that molecule. Specific, non-limiting examples of labels include fluorescent tags, enzymatic linkages, and radioactive isotopes.

Ligand Contact Residue or Specificity Determining Residue (SDR):

A residue within a CDR that is involved in contact with a ligand or antigen. A ligand contact residue is also known as a specificity determining residue (SDR). A non-ligand contact residue is a residue in a CDR that does not contact a ligand. A non-ligand contact residue can also be a framework residue.

Linkers:

covalent or very tight non-covalent linkages; chemical conjugation or direct gene fusions of various amino acid sequences, especially those (a) rich in Glycine Serine, Proline, Alanine, or (b) variants of naturally occurring linking amino acid sequences that connect immunoglobulin domains. Typical lengths may range from 5 up to 20 or more amino acids. The optimal lengths may vary to match the spacing and orientation of the specific target antigen(s), minimizing entropy but allowing effective binding of multiple antigens. Various arrangements are given in the figures.

Modification:

Changes to a protein sequence, structure, etc., or changes to a nucleic acid sequence, etc. As used herein, the term “modified” or “modification,” can include one or more mutations, deletions, substitutions, physical alteration (e.g., cross-linking modification, covalent bonding of a component, post-translational modification, e.g., acetylation, glycosylation, the like, or a combination thereof), the like, or a combination thereof. Modification, e.g., mutation, is not limited to random modification (e.g., random mutagenesis) but includes rational design as well.

Multimerizing Domain.

Many protein domains are known that form a very tight non-covalent dimer or multimer by associating with other protein domain(s). Some of the smallest examples are the so-called leucine zipper motifs, which are compact domains comprising heptad repeats that can either self-associate to form a homodimer (e.g. GCN4); alternatively, they may associate preferentially with another leucine zipper to form a heterodimer (e.g. myc/max dimers) or more complex tetramers (Chem. Biol. 2008 Sep. 22; 15(9):908-19. A heterospecific leucine zipper tetramer. Denq Y, Liu J, Zhenq Q, Li Q, Kallenbach N R, Lu M.). Closely related domains that have isoleucine in place leucine in the heptad repeats form trimeric “colied coil” assemblies (e.g. HIV gp41). Substitution of isoleucine for leucine in the heptad repeats of a dimer can alter the favoured structure to a trimer. Small domains have advantages for manufacture and maintain a small size for the whole protein molecule, but larger domains can be useful for multimer formation. Any domains that form non-covalent multimers could be employed. For example, the CH3 domains of IgG form homodimers, while CH1 and CL domains of IgG form heterodimers.

CH2D:

A CH2 or CH3 domain molecule engineered such that the molecule specifically binds antigen. The CH2 and CH3 domain molecules engineered to bind antigen are among the smallest known antigen-specific binding antibody domain-based molecules which can retain Fc receptor binding.

Neoplasia and Tumor:

The product of neoplasia is a neoplasm (a tumor), which is an abnormal growth of tissue that results from excessive cell division. Neoplasias are also referred to as “cancer.” A tumor that does not metastasize is referred to as “benign.” A tumor that invades the surrounding tissue and/or can metastasize is referred to as “malignant.” Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Nucleic Acid:

A polymer composed of nucleotide units (ribonucleotides, deoxyribonucleotides, related naturally occurring structural variants, and synthetic non-naturally occurring analogs thereof) linked via phosphodiester bonds, related naturally occurring structural variants, and synthetic non-naturally occurring analogs thereof. Thus, the term includes nucleotide polymers in which the nucleotides and the linkages between them include non-naturally occurring synthetic analogs, such as, for example and without limitation, phosphorothioates, phosphoramidates, methyl phosphonates, chiral-methyl phosphonates, 2′-O-methyl ribonucleotides, peptide-nucleic acids (PNAs), and the like. Such polynucleotides can be synthesized, for example, using an automated DNA synthesizer. The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.”

“cDNA” refers to a DNA that is complementary or identical to an mRNA, in either single stranded or double stranded form. “Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Recombinant nucleic acid” refers to a nucleic acid having nucleotide sequences that are not naturally joined together and can be made by artificially combining two otherwise separated segments of sequence. This artificial combination is often accomplished by chemical synthesis or, more commonly, by the artificial manipulation of isolated segments of nucleic acids, for example, by genetic engineering techniques. Recombinant nucleic acids include nucleic acid vectors comprising an amplified or assembled nucleic acid, which can be used to transform a suitable host cell. A host cell that comprises the recombinant nucleic acid is referred to as a “recombinant host cell.” The gene is then expressed in the recombinant host cell to produce a “recombinant polypeptide.” A recombinant nucleic acid can also serve a non-coding function (for example, promoter, origin of replication, ribosome-binding site and the like).

Operably Linked:

A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Pathogen:

A biological agent that causes disease or illness to its host. Pathogens include, for example, bacteria, viruses, fungi, protozoa and parasites. Pathogens are also referred to as infectious agents.

Examples of pathogenic viruses include those in the following virus families: Retroviridae (for example, human immunodeficiency virus (HIV); human T-cell leukemia viruses (HTLV); Picornaviridae (for example, polio virus, hepatitis A virus; hepatitis C virus; enteroviruses, human coxsackie viruses, rhinoviruses, echoviruses; foot-and-mouth disease virus); Calciviridae (such as strains that cause gastroenteritis); Togaviridae (for example, equine encephalitis viruses, rubella viruses); Flaviridae (for example, dengue viruses; yellow fever viruses; West Nile virus; St. Louis encephalitis virus; Japanese encephalitis virus; and other encephalitis viruses); Coronaviridae (for example, coronaviruses; severe acute respiratory syndrome (SARS) virus; Rhabdoviridae (for example, vesicular stomatitis viruses, rabies viruses); Filoviridae (for example, Ebola viruses); Paramyxoviridae (for example, parainfluenza viruses, mumps virus, measles virus, respiratory syncytial virus (RSV)); Orthomyxoviridae (for example, influenza viruses); Bunyaviridae (for example, Hantaan viruses; Sin Nombre virus, Rift Valley fever virus; bunya viruses, phleboviruses and Nairo viruses); Arena viridae (hemorrhagic fever viruses; Machupo virus; Junin virus); Reoviridae (e.g., reo viruses, orbiviurses and rotaviruses); Bimaviridae; Hepadnaviridae (Hepatitis B virus); Parvoviridae (parvoviruses); Papovaviridae (papilloma viruses, polyoma viruses; B K-virus); Adenoviridae (most adenoviruses); Herpesviridae (herpes simplex virus (HSV)-I and HSV-2; cytomegalovirus (CMV); Epstein-Barr virus (EBV); varicella zoster virus (VZV); and other herpes viruses, including HSV-6); Poxyiridae (variola viruses, vaccinia viruses, pox viruses); and Iridoviridae (such as African swine fever virus); Filoviridae (for example, Ebola virus; Marburg virus); Caliciviridae (for example, Norwalk viruses) and unclassified viruses (for example, the etiological agents of Spongiform encephalopathies, the agent of delta hepatitis (thought to be a defective satellite of hepatitis B virus); and astroviruses).

Examples of fungal pathogens include, but are not limited to: Cryptococcus neoformans, Histoplasma capsulatum, Coccidioides immitis, Blastomyces dermatitidis, Chlamydia trachomatis, Candida albicans.

Examples of bacterial pathogens include, but are not limited to: Helicobacter pylori, Borelia burgdorferi, Legionella pneumophilia, Mycobacteria species (such as M. tuberculosis, M. avium, M. intracellular, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species), Streptococcus pneumoniae, pathogenic Campylobacter species, Enterococcus species, Haemophilus influenzae, Bacillus anthracis, corynebacterium diphtheriae, corynebacterium species, Erysipelothrix rhusiopathiae, Clostridium perfringers, Clostridium tetani, Enterobacter aerogenes, Klebsiella pneumoniae, Pasturella multocida, Bacteroides species, Fusobacterium nucleatum, Streptobacillus moniliformis, Treponema pallidium, Treponema pertenue, Leptospira, and Actinomyces israelii.

Other pathogens (such as protists) include: Plasmodium falciparum and Toxoplasma gondii.

Pharmaceutically Acceptable Vehicles:

The pharmaceutically acceptable carriers (vehicles) useful in this disclosure may be conventional but are not limited to conventional vehicles. For example, E. W. Martin, Remington's Pharmaceutical Sciences, Mack Publishing Co., Easton, Pa., 15th Edition (1975) and D. B. Troy, ed. Remington: The Science and Practice of Pharmacy, Lippincott Williams & Wilkins, Baltimore Md. and Philadelphia, Pa., 21^{st} Edition (2006) describe compositions and formulations suitable for pharmaceutical delivery of one or more therapeutic compounds or molecules, such as one or more antibodies, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. As a non-limiting example, the formulation for injectable trastuzumab includes L-histidine HCl, L-histidine, trehalose dihydrate and polysorbate 20 as a dry powder in a glass vial that is reconstituted with sterile water prior to injection. Other formulations of antibodies and proteins for parenteral or subcutaneous use are well known in the art. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide:

A polymer in which the monomers are amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those that are recombinantly or synthetically produced. The term “residue” or “amino acid residue” includes reference to an amino acid that is incorporated into a protein, polypeptide, or peptide.

“Conservative” amino acid substitutions are those substitutions that do not substantially affect or decrease an activity or antigenicity of a polypeptide. For example, a polypeptide can include at most about 1, at most about 2, at most about 5, at most about 10, or at most about 15 conservative substitutions and specifically bind an antibody that binds the original polypeptide. The term conservative variation also includes the use of a substituted amino acid in place of an unsubstituted parent amino acid, provided that antibodies raised antibodies raised to the substituted polypeptide also immunoreact with the unsubstituted polypeptide. Examples of conservative substitutions include: (i) Ala-Ser; (ii) Arg-Lys; (iii) Asn-Gin or His; (iv) Asp-Glu; (v) Cys-Ser; (vi) Gin-Asn; (vii) Glu-Asp; (viii) His-Asn or Gln; (ix) Ile-Leu or Val; (x) Leu-Ile or Val; (xi) Lys-Arg, Gln, or Glu; (xii) Met-Leu or Ile; (xiii) Phe-Met, Leu, or Tyr; (xiv) Ser-Thr; (xv) Thr-Ser; (xvi) Trp-Tyr; (xvii) Tyr-Trp or Phe; (xviii) Val-Ile or Leu.

Conservative substitutions generally maintain (a) the structure of the polypeptide backbone in the area of the substitution, for example, as a sheet or helical conformation, (b) the charge or hydrophobicity of the molecule at the target site, and/or (c) the bulk of the side chain. The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Preventing, Treating, Managing, or Ameliorating a Disease:

“Preventing” a disease refers to inhibiting the full development of a disease. “Treating” refers to a therapeutic intervention that ameliorates a sign or symptom of a disease or pathological condition after it has begun to develop. “Managing” refers to a therapeutic intervention that does not allow the signs or symptoms of a disease to worsen. “Ameliorating” refers to the reduction in the number or severity of signs or symptoms of a disease.

Probes and Primers:

A probe comprises an isolated nucleic acid attached to a detectable label or reporter molecule. Primers are short nucleic acids, and can be DNA oligonucleotides 15 nucleotides or more in length, for example. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example, by the polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides.

Purified:

The term purified does not require absolute purity; rather, it is intended as a relative term. Thus, for example, a purified CH2 or CH3 domain molecule is one that is isolated in whole or in part from naturally associated proteins and other contaminants in which the molecule is purified to a measurable degree relative to its naturally occurring state, for example, relative to its purity within a cell extract or biological fluid.

The term “purified” includes such desired products as analogs or mimetics or other biologically active compounds wherein additional compounds or moieties are bound to the CH2 or CH3 domain molecule in order to allow for the attachment of other compounds and/or provide for formulations useful in therapeutic treatment or diagnostic procedures.

Generally, substantially purified CH2 or CH3 domain molecules include more than 80% of all macromolecular species present in a preparation prior to admixture or formulation of the respective compound with additional ingredients in a complete pharmaceutical formulation for therapeutic administration. Additional ingredients can include a pharmaceutical carrier, excipient, buffer, absorption enhancing agent, stabilizer, preservative, adjuvant or other like co-ingredients. More typically, the CH2 or CH3 domain molecule is purified to represent greater than 90%, often greater than 95% of all macromolecular species present in a purified preparation prior to admixture with other formulation ingredients. In other cases, the purified preparation may be essentially homogeneous, wherein other macromolecular species are less than 1%.

Recombinant:

A recombinant nucleic acid or polypeptide is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, for example, by genetic engineering techniques. Recombinant proteins may be made in cells transduced with genetic elements to direct the synthesis of the heterologous protein. They may also be made in cell-free systems. Host cells that are particularly useful include mammalian cells such as CHO and HEK 293, insect cells, yeast such as Pichia pastoris or Saccharomyces, or bacterial cells such as E. coli or Pseudomonas.

Sample:

A portion, piece, or segment that is representative of a whole. This term encompasses any material, including for instance samples obtained from a subject.

A “biological sample” is a sample obtained from a subject including, but not limited to, cells, tissues and bodily fluids. Bodily fluids include, for example, saliva, sputum, spinal fluid, urine, blood and derivatives and fractions of blood, including serum and lymphocytes (such as B cells, T cells and subfractions thereof). Tissues include those from biopsies, autopsies and pathology specimens, as well as biopsied or surgically removed tissue, including tissues that are, for example, unfixed, frozen, fixed in formalin and/or embedded in paraffin.

In some embodiments, a biological sample is obtained from a subject, such as blood or serum. A biological sample is typically obtained from a mammal, such as a rat, mouse, cow, dog, guinea pig, rabbit, or primate. In some embodiments, the primate is macaque, chimpanzee, or a human.

Scaffold:

In some embodiments, a CH2 or CH3 domain scaffold is a recombinant CH2 or CH3 domain that can be used as a platform to introduce mutations (such as into the loop regions) in order to confer antigen binding to the CH2 or CH3 domain. In some embodiments, the scaffold is altered to exhibit increased stability compared with the native CH2 or CH3 domain. In particular examples, the scaffold is mutated to introduce pairs of cysteine residues to allow formation of one or more non-native disulfide bonds. In some cases, the scaffold is a CH2 or CH3 domain having an N-terminal deletion, such as a deletion of about 1 to about 7 amino acids. Scaffolds are not limited to these definitions.

Sequence Identity:

The similarity between nucleotide or amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, Journal of Molecular Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids Research 16:10881-10890, 1988; and Pearson and Lipman, Proc. Natl. Acad. Sci. U.S.A. 85:2444, 1988. Altschul et al., Nature Genetics 6:119-129, 1994.

The NCBI Basic Local Alignment Search Tool (BLAST™) (Altschul et al., Journal of Molecular Biology 215:403-410, 1990.) is available from several sources, including the National Center for Biotechnology Information (NCBI, Bethesda, Md.) and on the Internet, for use in connection with the sequence analysis programs blastp, blastn, blastx, tblastn and tblastx.

Specific Binding Agent:

An agent that binds substantially only to a defined target. Thus an antigen specific binding agent is an agent that binds substantially to an antigenic polypeptide or antigenic fragment thereof. In one embodiment, the specific binding agent is a monoclonal or polyclonal antibody or a CH2 or CH3 domain molecule that specifically binds the antigenic polypeptide or antigenic fragment thereof.

The term “specifically binds” refers to the preferential association of a binding agent, such as a CH2D or other ligand molecule, in whole or part, with a cell or tissue bearing that target of that binding agent and not to cells or tissues lacking a detectable amount of that target. It is, of course, recognized that a certain degree of non-specific interaction may occur between a molecule and a non-target cell or tissue. Nevertheless, specific binding may be distinguished as mediated through specific recognition of the antigen. Specific binding results in a much stronger association between the CH2 or CH3 domain molecule and cells bearing the target molecule than between the bound or CH2 or CH3 domain molecule and cells lacking the target molecule. Specific binding typically results in greater than 2-fold, such as greater than 5-fold, greater than 10-fold, or greater than 100-fold increase in amount of bound CH2 or CH3 domain molecule (per unit time) to a cell or tissue bearing the target polypeptide as compared to a cell or tissue lacking the target polypeptide, respectively. Specific binding to a protein under such conditions requires aCH2 or CH3 domain molecule that is selected for its specificity for a particular protein. A variety of immunoassay formats are appropriate for selecting CH2 or CH3 domain molecules specifically reactive with a particular protein. For example, solid-phase ELISA immunoassays are routinely used.

Subject:

Living multi-cellular organisms, including vertebrate organisms, a category that includes both human and non-human mammals.

Therapeutically Effective Amount:

A quantity of a specified agent sufficient to achieve a desired effect in a subject being treated with that agent. Such agents include the CH2 or CH3 domain molecules described herein. For example, this may be the amount of an H1V-specific CH2 domain molecule useful in preventing, treating or ameliorating infection by HIV. Ideally, a therapeutically effective amount of a CH2D is an amount sufficient to prevent, treat or ameliorate infection or disease, such as is caused by HIV infection in a subject without causing a substantial cytotoxic effect in the subject. The therapeutically effective amount of an agent useful for preventing, ameliorating, and/or treating a subject will be dependent on the subject being treated, the type and severity of the affliction, and the manner of administration of the therapeutic composition.

Toxin:

A molecule that is cytotoxic for a cell. Toxins include, but are not limited to, abrin, ricin, Pseudomonas exotoxin (PE), diphtheria toxin (DT), botulinum toxin, saporin, restrictocin or gelonin, or modified toxins thereof. For example, PE and DT are highly toxic compounds that typically bring about death through liver toxicity. PE and DT, however, can be modified into a form for use as an immunotoxin by removing the native targeting component of the toxin (for example, domain Ia of PE or the B chain of DT) and replacing it with a different targeting moiety, such as a CH2 or CH3 domain molecule. Toxins may also include small molecule toxins. (See the definition of immunoconjugates.)

Transduced:

A transduced cell is a cell into which has been introduced a nucleic acid molecule by molecular biology techniques. As used herein, the term transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Tumor-Associated Antigens (TAAs):

A tumor antigen which can stimulate tumor-specific T-cell-defined immune responses. Exemplary TAAs include, but are not limited to, RAGE-I, tyrosinase, MAGE-1, MAGE-2, NY-ESO-I, Melan-A/MART-1, glycoprotein (gp) 75, gplOO, beta-catenin, PRAME, MUM-I, WT-I, CEA, and PR-1. Additional TAAs are known in the art (for example see Novellino et al., Cancer Immunol. Immunother. 54(3): 187-207, 2005) and includes TAAs not yet identified. Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. Viral-associated antigen (VAAs): A viral antigen which can stimulate viral-specific T-cell-defined immune responses. Exemplary VAAs include, but are not limited to, an antigen from human immunodeficiency virus (HIV), BK virus, JC virus, Epstein-Barr virus (EBV), cytomegalovirus (CMV), adenovirus, respiratory syncytial virus (RSV), herpes simplex virus 6 (HSV-6), parainfluenza 3, or influenza B.

### BRIEF DESCRIPTION OF THE DRAWINGS

FIG. 1 is a schematic representation of various embodiments of multimer CH2D molecules of the present invention, for example, two different multimer CH2Ds each comprising a first CH2 domain and a second CH2 domain (FIG. 1A, FIG. 1B); a multimer CH2D comprising a first CH2D, a second CH2D, and a third CH2D (FIG. 1C); and a multimer CH2D comprising a first CH2D, a second CH2D, a third CH2D, a fourth CH2D, and a fifth CH2D (FIG. 1D). FIG. 1 shows the CH2Ds being linked via linkers. FIG. 1 also shows target binding regions of the CH2Ds. Target binding regions may include but are not limited to one or more CDRs or fragments thereof, modified loops (or portions thereof) of the CH2Ds having specificity for the target (e.g., comprising one or more CDRs or fragments thereof), and the like.

FIG. 2 is a schematic representation of various embodiments of CH2D multimers of the present invention, for example linkers comprising multimerizing domains. In some embodiments, two or more CH2Ds are linked via the multimerizing domains of the linkers.

FIG. 3 is a schematic representation of an embodiment of a CH2D multimer of the present invention wherein a first CH2D is linked to a second CH2D via hinge components (comprising multimerizing domains). For example, the first CH2D may comprise a first half hinge component (with a first multimerizing domain) and the second CH2D may comprise a second half hinge component (with a second multimerizing domain). In this example, the multimerizing domains are linked to the respective hinge components via a site capable of being cleaved by a protease. As shown in FIG. 3, proteolytic cleavage of the hinge components removes the multimerizing domains from the CH2D multimer, resulting in a “hinge dimer.”

FIG. 4 is a schematic representation of various embodiments of CH2D multimers of the present invention conferring specificity for two targets. FIG. 4A illustrates a multimer comprising of a first CH2D (left), a second CH2D (middle), and a third CH2D (right), wherein the first and second CH2D each comprise a target binding region specific for a first target, while the third CH2D comprises a target binding region specific for a second (different) target. FIG. 4B illustrates a CH2D multimer comprising a first CH2D (left) linked to a second CH2D (right) via hinge components (the hinge components comprising multimerizing domains). The first CH2D has a target binding region specific for a first target and the second CH2D has a target binding region specific for a second target.

FIG. 5 is a schematic representation of various embodiments of CH2D multimers of the present invention comprising one or more FcRn binding sites. FIG. 5A illustrates an example of a CH2D multimer comprising three CH2Ds, each CH2D comprising a FcRn receptor. FIG. 5B illustrates an example of a CH2D multimer comprising two CH2Ds linked via a hinge component, both CH2Ds comprising a FcRn receptor.

FIG. 6 is schematic representation of various embodiments of CH2D multimers comprising of the present invention having one or more F_{c}γ receptor binding sites. FIG. 6A shows a CH2Dmultimer comprising three CH2Ds, each comprising an F_{c}γ receptor binding site (e.g., unmodified or modified). FIG. 6B shows a CH2D multimer comprising three CH2Ds, wherein only one CH2 domain comprises a F_{c}γ receptor binding site (e.g., unmodified or modified).

FIG. 7 shows that the stability of CH2, m01 and dimer CH2 were assessed in cynomolgus serum incubated at 37° C. from 0 to 7 days. Serum samples were subjected to SDS-PAGE followed by Western Blotting. The left panel is the native single domain isolated CH2 (CH2D); the middle panel is engineered CH2 (m01); and the right panel is a dimer of the native CH2 (dimer CH2) protein.

FIG. 8A shows the amino acid sequence alignment of wild-type CH2, m01 and m01s. FIG. 8B shows the comparison of the expression of CH2, m01 and m01s. FIG. 8C shows size exclusion chromatography was used to assess whether m01s existed as a monomer or dimer in PBS at pH 7.4. The insert is a standard curve.

FIG. 9 shows the measurement of the Tm value of m01s. The Tm values (68.9° C., 65.7° C. 63.6° C. and 59.3° C. correspond to 3 M, 3.5 M, 4 M and 5 M Urea, respectively) from Circular Dichroism. The calculated Tm for m01s in 0 M Urea is 82.6° C.

FIG. 10 shows HIS-CH2D and HIS-m01s were expressed in and purified from E. coli by Blue Sky BioServices using small-scale 1 L preparations. The purified protein preparations were subjected to SDS-PAGE, and the gels were stained with Coomasie blue. The bands corresponding to HIS-CH2D (right panel) and HIS-m01s (left panel) are indicated by the arrows. The yields of protein are indicated.

FIG. 11 shows HIS-CH2D and HIS-m01s were expressed in and purified from E. coli by Blue Sky BioServices using large-scale 10 L preparations. The samples were subjected to SDS-PAGE, and the gels were stained with Coomasie blue. The bands corresponding to HIS-CH2D (upper) and HIS-m01s (lower) are indicated by the arrows.

FIG. 12 shows sequences for CH2D constructs that will be commercially produced by Blue Sky BioServices and tested at SFBR in primate studies.

FIG. 13 shows design of different CH2D constructs with an additional cysteine and hinge region from IgG at N- or C-terminal.

FIG. 14 shows the CH2D construct HIS-GSGS-hinge6-CH2 was produced in and purified from E. coli. The protein preparations were subjected to size exclusion chromatography (a), and 10 mL fractions were obtained and subjected to SDS-PAGE under both non-reducing (b, c) and reducing conditions (d, e). The gels were stained with Coomasie blue. The band corresponding to HIS-GSGS-hinge6-CH2 is indicated in each gel by the arrow.

FIG. 15 shows estimation of dimer formation of CH2D constructs with an additional cysteine and hinge region from IgG at the N- or C-terminal of CH2D. The insert is a standard curve.

FIG. 16 shows design of different constructs with two CH2 domains connected by different string linkers.

FIG. 17 shows estimation of the molecular weight of the constructs with two CH2 domains connected by different string linkers. The same standard curve as in FIG. 15 is used.

FIG. 18 shows design of two m01 constructs with an additional cysteine and hinge region from IgG at the N- or C-terminal.

FIG. 19 shows estimation of dimer formation of m01 constructs with an additional cysteine and hinge region from IgG at the N- or C-termini. The same standard curve as in FIG. 15 is used.

FIG. 20 shows binding of CH2, m01, m01s, Fc, VH domain, ScFv on yeast cells to FcRn at pH7.4 (black) and pH6.0 (grey). Anti-CH2 antibody and anti-c-Myc antibody were used to detect the expression of the CH2Ds, and PE-streptavidin was used as negative control.

FIG. 21 shows binding of m01s to FcRn. A. Binding of m01s to FcRn at different FcRn concentrations (0, 2.5, 5, 10, 20, 50 and 100 nM) at pH 6.0. B. Inhibition of binding of m01s to FcRn on the surface of yeast cells by IgG at different IgG concentrations (0, 0.125, 0.5 and 4 μM).

FIG. 22 shows schematic of library construction based on m01s scaffold.

FIG. 23 shows binding of B2 (▪) to sp62 and related peptides with positive control 2F5 (▴) and negative control m01s (). A. Binding of B2, m01s and 2F5 to sp62. B. Binding of B2, m01s and 2F5 to sp62 scrambled peptide. 2F5 showed non-specific binding signal while B2 did not exhibit binding to the scrambled peptide.

FIG. 24 shows neutralization activities of B2 (5 μM) and B2 mutant 2 (5 μM). The cell line-based assay was carried out in HOS CD4+CCR5+ target cells containing a tat-inducible luciferase reporter that express CD4, CCR5. Infectivity titers were determined on the basis of luminescence measurements at 3 days post-infection of the cells by pseudotyped viruses. Neutralization assays were carried out in triplicate wells by preincubation of the antibodies with pseudotype viruses for 30 min at 37° C. followed by infection of 1−2×10⁴ HOS CD4+CCR5+ cells. The degree of virus neutralization by antibody was achieved by measuring luciferase activity. Luminescence was measured after 3 days. The mean luminescence readings for triplicate wells were determined.

FIG. 25 shows polyclonal phage ELISA for testing panning result after three-round panning. After three round panning, polyclonal phage ELISA was used for estimation of the enrichment. 50 μl 2 μg/ml NCL per well was coated. BSA was also coated as negative control. 5×10¹⁰ phage from each round panning was added to the wells. HRP-anti-M13 antibody was used for detection of phage.

FIG. 26 shows the binding of CH2-derived monomers and homodimers, and control proteins (scFv m9, m36, dimer m36 and BSA) to gp140a (2 ug/ml) in the presence of soluble CD4 (2 ug/ml) was assessed using ELISA.

FIG. 27 shows the binding of CH2-derived monomers and homodimers, and control proteins (scFv m9, m36, dimer m36 and BSA) to gp140b (2 ug/ml) in the presence of soluble CD4 (2 ug/ml) was assessed using ELISA.

FIG. 28 shows the binding of CH2-derived monomers and homodimers, and control proteins (scFv m9, m36, dimer m36 and BSA) to gp140c (2 ug/ml) in the presence of soluble CD4 (2 ug/ml) was assessed using ELISA.

FIG. 29 shows the binding of CH2-derived monomers and heterodimers, and control proteins (m36 and BSA) to gp140c, which was assessed using ELISA.

FIG. 30 shows the binding of CH2-derived monomers and heterodimers, and control proteins (m36 and BSA) to gp140c in the presence of soluble CD4 (2 ug/ml), which was assessed using ELISA.

FIG. 31 shows TABLE 1 which lists all the CH2D Monomers and Dimers Produced in and Purified from E. coli.

FIG. 32 shows TABLE 2 which summarizes all the linkers tested.

FIG. 33 shows TABLE 3 which provides the results from an ELISA testing the binding of monomer and homodimer CH2Ds to gp140a. The values correspond to the OD at 405 nm.

FIG. 34 shows TABLE 4 which provides the results from an ELISA testing the binding of monomer and homodimer CH2Ds to gp140b. The values correspond to the OD at 405 nm.

FIG. 35 shows TABLE 5 which provides the results from an ELISA testing the binding of monomer and homodimer CH2Ds to gp140c. The values correspond to the OD at 405 nm.

FIG. 36 shows TABLE 6 which provides the results from an ELISA testing the binding of monomers and heterodimer CH2Ds to gp140c. The values correspond to the OD at 405 nm.

FIG. 37 shows TABLE 7 which provides the results from an ELISA testing the binding of monomer and heterodimer CH2Ds to SCgp140c. The values correspond to the OD at 405 nm.

FIG. 38 shows TABLE 8 which provides for non-limiting examples of CH2 domain fragments.

FIG. 39 shows TABLE 9 which provides for non-limiting examples of CH2 domains with deletions.

FIG. 40 shows TABLE 10 which provides for non-limiting examples of CH2 domains with substitutions.

### DESCRIPTION OF PREFERRED EMBODIMENTS

As used herein, the term “CH2 domain” or “CH2D” refers to a CH2 domain of IgG, IgA, or IgD, or a fragment thereof; a peptide domain substantially resembling a CH2 domain of IgG, IgA or IgD or a fragment thereof; or peptide domain functionally equivalent to a CH2 domain of IgG, IgA, IgD, or a fragment thereof, for example a CH3 domain of IgE or IgM, or a fragment thereof. Non-limiting examples of fragment of a CH2 domain and peptide domain substantially resembling a CH2 domain are fully disclosed herein below under the heading “CH2 DOMAIN MODIFICATIONS”.

### Multimeric CH2Ds

The present invention features multimeric CH2D proteins. In some embodiments, a CH2D multimer comprises at least two CH2 domains (CH2 immunoglobulin domains), for example the CH2D multimer is a dimer comprising a first CH2 domain and a second CH2 domain. Or, the CH2D multimer may be a trimer comprising a first CH2 domain, a second CH2 domain, and a third CH2 domain. In some embodiments, the CH2D multimer may be a tetramer comprising a first CH2D, a second CH2 domain, a third CH2 domain, and a fourth CH2 domain. In some embodiments, the CH2D multimer may be a pentamer comprising a first CH2 domain, a second CH2 domain, a third CH2 domain, a fourth CH2 domain, and a fifth CH2 domain. In some embodiments, the CH2D multimer may be a hexamer comprising a first CH2 domain, a second CH2 domain, a third CH2 domain, a fourth CH2 domain, a fifth CH2 domain, and a sixth CH2 domain. In some embodiments, the CH2D multimer comprises more than six CH2 domains.

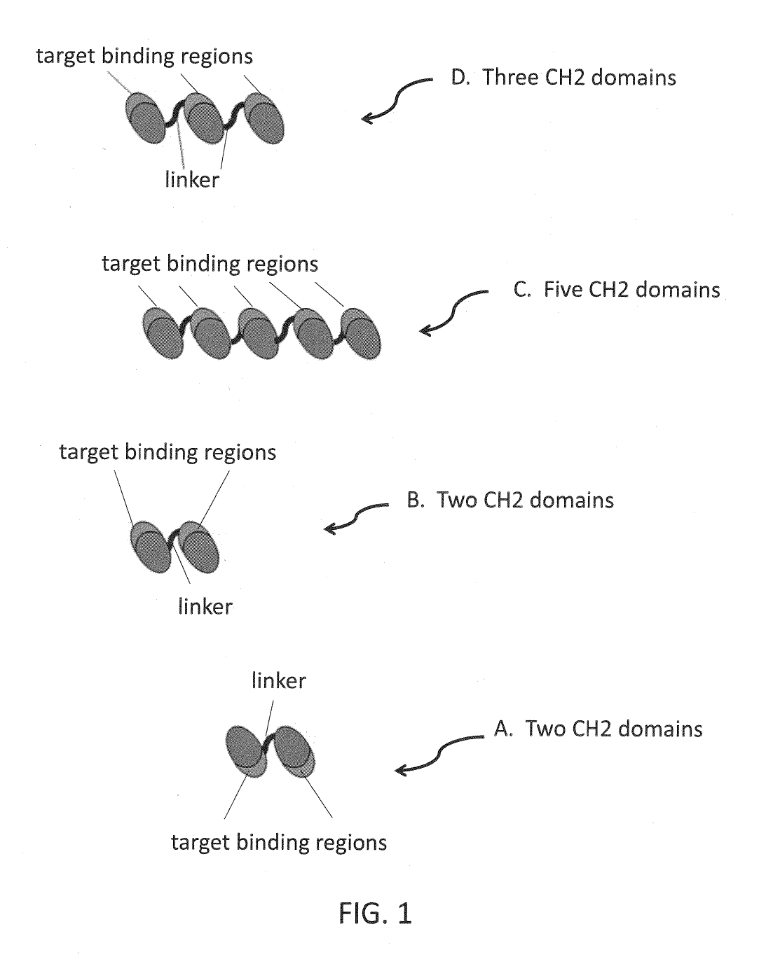

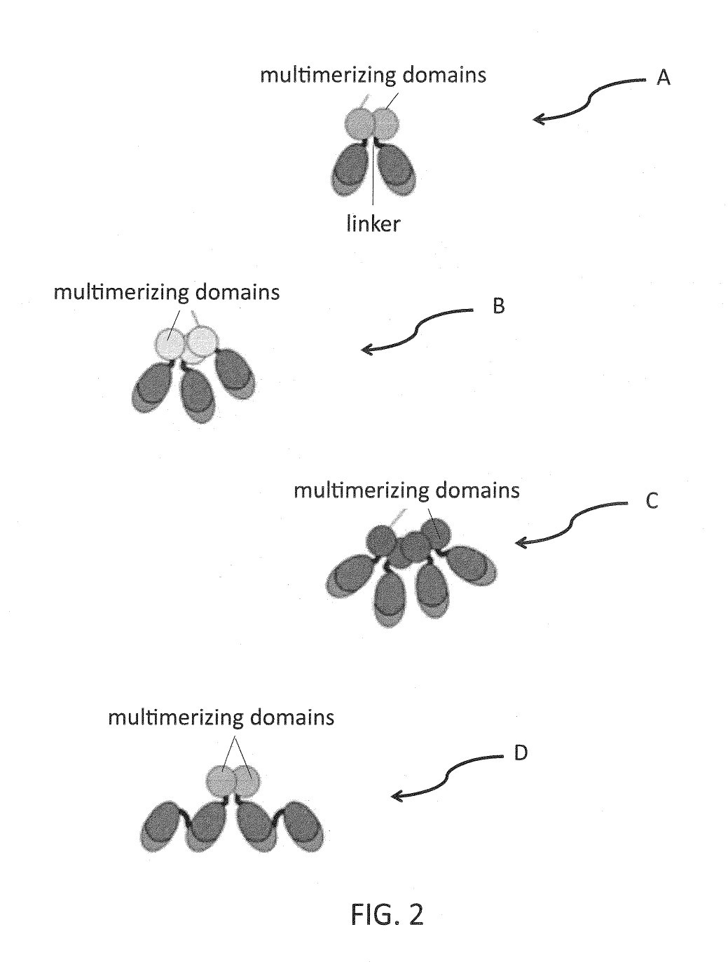

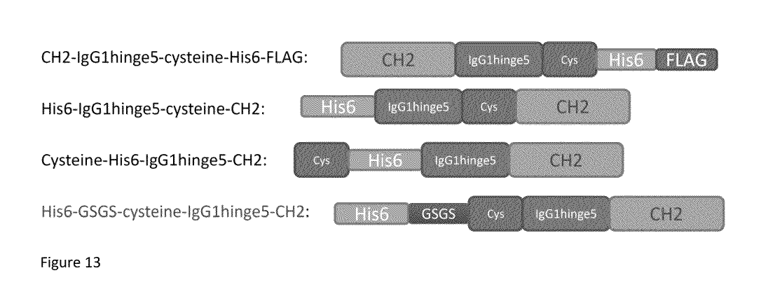

Two CH2 domains may be coupled by a linker, wherein the linker can be attached to the individual CH2 domain at any appropriate location on the CH2 domain. Examples of where a linker may attach onto the CH2 domain include the following location on the CH2 domain: the carboxy terminus, the amino-terminus, a cysteine preceding or following the carboxy-terminus or amino-terminus of the CH2 domain (see for example, FIGS. 13, 16, and 18). In some embodiments, a linking of two or more CH2 domains (e.g., to form a dimer, a trimer, etc.) is driven by the formation of a disulfide bond between the cysteines at the carboxy or amino-terminus of the CH2Ds and via the introduction of the linker (FIGS. 1, 13, 16, and 18). The formation of CH2D multimers in solution can be monitored using size exclusion chromatography: therefore, this enables the dimerization potential of the linker to be assessed (FIGS. 8c, 15, and 18). In addition, a CH2 domain and a multimerizing domain can be coupled by a linker (FIG. 2d); this leads to aggregation of the CH2 domains.

In some embodiments, a linker may be selected from the group consisting of 2-iminothiolane, N-succinimidyl-3-(2-pyridyldithio) propionate (SPDP), 4-succinimidyloxycarbonyl-alpha-(2-pyridyldithio)toluene (SMPT), m-maleimidobenzoyl-N-hydroxysuccinimide ester (MBS), N-succinimidyl (4-iodoacetyl)aminobenzoate (SIAB), succinimidyl 4-(p-maleimidophenyl)but-yrate (SMPB), 1-ethyl-3-(3-dimethylaminopropyl)carbodiimide (EDC), bis-diazobenzidine and glutaraldehyde. In some embodiments, a linker may be attached to an amino group, a carboxylic group, a sulfhydryl group or a hydroxyl group of an amino acid group of the CH2 domain. As an example only, SEQ ID NO. 1 shown in FIG. 8A is an amino acid sequence of a CH2 domain. The amino group that a linker may attach to include, for example, alanine, lysine, or proline. The carboxylic group that a linker may be attached to may be, for example, aspartic acid (D82, D40), glutamic acid (E3, E39). The sulfhydryl group that a linker may be attached to may be, for example, cysteine (C31, C91). The hydroxyl group that a linker may be attached to may be, for example, serine (S9), threonine (T30), or tyrosine (Y70). For example, a linker may be linked to a carboxyl acid group of amino acid of the CH2 domain. Although the described chemistry may be used to couple the CH2 domains of the described invention, any other coupling chemistry known to those skilled in the art capable of chemically attaching a CH2 domain to another CH2 domain or multimerizing domain of the invention is covered by the scope of this invention.

As discussed previously, the CH2 domain may include a CH2 domain of IgG, IgA, IgD, a fragment of a CH2 domain of IgG, IgA, IgD, or a CH2-like domain, for example an immunoglobulin domain that substantially resembles a CH2 domain of IgG, IgA, or IgD. Domains that substantially resemble a CH2 domain of IgG, IgA, or IgD may include but are not limited to a CH3 domain of IgE or IgM, or fragments thereof.

In some embodiments, the first CH2 domain (CH2 immunoglobulin domain) of the CH2D multimer is a CH2 domain of IgG, IgA, or IgD, or a CH3 domain of IgE or IgM, or a fragment thereof. In some embodiments, the second immunoglobulin CH2 domain of the multimer is a CH2 domain of IgG, IgA, or IgD, or a CH3 domain of IgE or IgM, or a fragment thereof. Like the first and second CH2 domain, the third CH2 domain, fourth CH2 domain, fifth CH2 domain, and/or sixth CH2 domain may be a CH2 domain of IgG, IgA, or IgD, or a CH3 domain of IgE or IgM, or a fragment thereof, or any combination thereof.

Briefly, whole immunoglobulins comprise two light chains, each having a variable domain and a constant domain, and two heavy chains, each having a variable domain and either three or four constant domains. In some embodiments, the multimeric CH2D of the present invention is substantially free of an immunoglobulin CH1 domain. In some embodiments, the multimeric CH2D is substantially free of a CH3 domain derived from IgG, IgA, or IgD, or a CH4 domain derived from IgM or IgE. The multimeric CH2D may be substantially free of a constant light (CL) domain. The CH2D multimer may be substantially free of an entire immunoglobulin variable domain, for example a VH domain or a VL domain. However, in some embodiments, the CH2 multimer comprises a portion of a variable domain (e.g., VH domain, VL domain).

### CH2 Domain Modifications

Each domain in an immunoglobulin has a conserved structure referred to as the immunoglobulin fold. The immunoglobulin fold comprises two beta sheets arranged in a compressed anti-parallel beta barrel. With respect to constant domains, the immunoglobulin fold comprises a 3-stranded sheet containing strands C, F, and G, packed against a 4-stranded sheet containing strands A, B, D, and E. The strands are connected by loops. The fold is stabilized by hydrogen bonding, by hydrophobic interactions, and by a disulfide bond. In some embodiments, the CH2Ds may be stabilized by the incorporation of additional disulfide bonds. With respect to variable domains, the immunoglobulin fold comprises a 4-stranded sheet containing strands A, B, D, and E, and a 5-stranded sheet containing strands C, F, G, C′, and C″.

The variable domains of both the light and heavy chains contain three complementarity-determining regions (CDRs): CDR1, CDR2, and CDR3. The CDRs are loops that connect beta strands of the immunoglobulin folds, for example B-C, C′-C″, and F-G. The residues in the CDRs regulate antigen specificity and/or affinity.

The CH2D multimer may effectively bind to a target antigen (or one or more target antigens). In some embodiments, the CH2D multimer has a greater avidity and/or affinity for the target (or targets) as compared to the avidity and/or affinity of a monomer derived from the CH2D multimer or a comparable antibody.

In some embodiments the CH2D multimer comprises at least one CDR (e.g., CDR1, CDR2, CDR3) or a functional fragment thereof. For example, the CH2D multimer may comprise one, two, three, or more CDRs or functional fragments thereof. Some or all of the CDRs or functional fragments thereof may be identical peptides or different peptides. The CDRs or functional fragments thereof may be associated with the first CH2 domain and/or second CH2 domain. In some embodiments, in the case of a protein comprising three or more CH2 domains, the CDRs or functional fragments thereof may be associated with the first CH2 domain and/or the second CH2 domain and/or the third CH2 domain and/or the fourth CH2 domain and/or the fifth CH2 domain and/or the sixth CH2 domain, etc.

One or more loops and/or strands (of the beta sheets, A, B, C, D, E, F, G) of one or more CH2 domains may be modified. As used herein, the term “modified” or “modification,” can include one or more mutations, deletions, substitutions, physical alteration (e.g., cross-linking modification, covalent bonding of a component, post-translational modification, e.g., acetylation, glycosylation, the like, or a combination thereof), the like, or a combination thereof. Modification, e.g., mutation, is not limited to random modification (e.g., random mutagenesis) but includes rational design as well.

In some embodiments, a loop (or a portion thereof) of a CH2 domain (e.g., the first CH2 domain, the second CH2 domain, etc.) is modified, e.g., entirely or partially replaced with a CDR (e.g., CDR1, CDR2, CDR3) or a functional fragment thereof, mutated, deleted, substituted, etc. Loops refer to portions of the protein between the strands of the beta sheets (e.g., A, B, C, D, E, F, G). Loops may include, for example, Loop 1, Loop 2, or Loop 3, A-B, Loop C-D, or Loop E-F. In some embodiments, a strand (e.g., A, B, C, D, E, F, G) or a portion thereof of a CH2 domain (e.g., the first CH2 domain, the second CH2 domain, etc.) is modified, e.g., entirely or partially replaced with a CDR (e.g., CDR1, CDR2, CDR3) or a functional fragment thereof, mutated, deleted, substituted, etc. In some embodiments, a strand (e.g., A, B, C, D, E, F, G) or a portion thereof and a loop or a portion thereof of a CH2 domain are modified, e.g., entirely or partially replaced with one CDR (e.g., CDR1, CDR2, CDR3), a functional fragment thereof, more than one CDR (e.g., CDR1, CDR2, CDR3), or one or more functional fragments thereof, mutated, deleted, substituted, etc. See, for example, Tables 8, 9 and 10 for additional examples of CH2 domain fragments, CH2 domain with deletions and CH2 domain with substitution(s)/mutation(s).

In some embodiments, more than one loop (or portions thereof) of a CH2 domain of the multimer may be modified, e.g., entirely or partially replaced with one or more CDRs or a functional fragment thereof, mutated, deleted, substituted, etc. In some embodiments, one or more loops (or portions thereof) of more than one CH2 domain (e.g., first CH2 domain and second CH2 domain) may be modified, e.g., entirely or partially replaced with one or more CDRs (e.g., CDR1, CDR2, CDR3), or one or more functional fragments thereof, mutated, deleted, substituted, etc.

In some embodiments, Loop 1 of the first CH2 domain and/or second CH2 domain is modified, for example Loop 1 is entirely or partially replaced by one or more CDRs or one or more fragments thereof, is mutated, is deleted, substituted, and/or the like. In some embodiments, Loop 2 of the first CH2 domain and/or second CH2 domain is modified, for example Loop 1 is entirely or partially replaced by one or more CDRs or one or more fragments thereof, is mutated, is deleted, and/or the like. Likewise, in some embodiments, Loop 3 and/or Loop A-B and/or Loop C-D and/or Loop E-F is modified, for example entirely or partially replaced by one or more CDRs or one or more fragments thereof, mutated, deleted, and/or the like. In the case of a CH2D multimer comprising more than two CH2 domains, Loop 1, Loop 2, Loop 3, Loop A-B, Loop C-D, and/or Loop E-F may be modified (e.g., entirely or partially replaced by one or more CDRs or one or more fragments thereof, mutated, deleted, and/or the like).

The loops and/or strands of the CH2 domains are not always modified with a CDR or fragment thereof. Other peptide sequences may be used to modify (e.g., substitute, replace, etc.) loops and/or strands of one or more CH2 domains.

The CH2 domain may comprise deletions, e.g., deletions of portions of the N-terminus and/or portions of the C-terminus. In some embodiments, the deletion may be between about 1 to 10 amino acids. For example, in some embodiments, the CH2 domain comprises a deletion of the first seven amino acids of the N-terminus. Or, in some embodiments, the CH2 domain comprises a deletion of the first amino acid, the first two, the first three, the first four, the first five, or the first six amino acids of the N-terminus. In some embodiments, the CH2 domain comprises a deletion of the first eight, the first nine, or the first ten amino acids of the N-terminus. In some embodiments, the CH2 domain comprises a deletion of the last four amino acids of the C-terminus. In some embodiments, the CH2 domain comprises a deletion of the last amino acid, the last two, or the last three amino acids of the C-terminus. The present invention is not limited to the aforementioned examples of deletions. The CH2 domain may comprise other deletions in other regions of the protein.

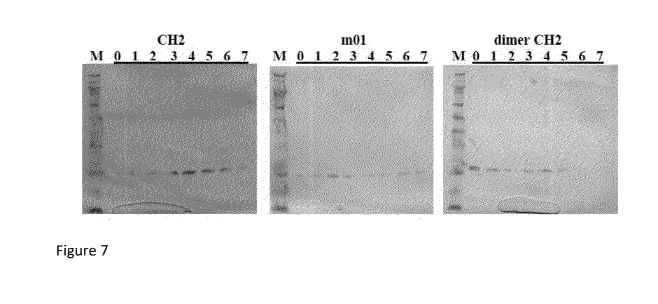

One or more portions of the CH2 domain or one or more amino acids may be substituted with another peptide or amino acid, respectively. For example, in some embodiments, the CH2 domain comprises a first amino acid substitution. In some embodiments, the CH2 domain comprises a first amino acid substitution and a second amino acid substitution. In some embodiments, the CH2 domain comprises a first amino acid substitution, a second amino acid substitution, and a third amino acid substitution. Examples of amino acid substitutions may include but is not limited to V10 TO C10, L12 to C12 (FIG. 8A, m01 and m01s), and/or K104 to C104 (FIG. 8A, m01 and m01s). Substitutions may in some cases confer increased protein stability among other properties (m01s, FIG. 7).

As non-limiting examples, a fragment of a CH2 domain includes: a CH2 domain without a first amino acid at the N-terminus as compared to a native CH2 domain, a CH2 domain without up to the first 10 amino acid at the N-terminus as compared to a native CH2 domain, a CH2 domain without a first amino acid at the C-terminus as compared to a native CH2 domain, or a CH2 domain without up to the first 10 amino acid at the C-terminus as compared to a native CH2 domain.

As a non-limiting example, a peptide domain substantially resembling a CH2 domain of IgG may include a CH2 domain of IgG comprising at least one amino acid substitution or deletion.

### Single or Multiple Target Specificity

The CH2D multimers of the present invention may be specific for one or more targets. For example, one or more CH2 domains of the multimer may be directed to a first target while one or more other CH2 domains of the multimer may be directed to a second target. In some embodiments, the first immunoglobulin CH2 domain and the second immunoglobulin CH2 domain are both specific for a first target. In some embodiments, the first immunoglobulin CH2 domain is specific for a first target and the second immunoglobulin CH2 domain is specific for a second target.

The CH2D multimer may be directed against a single target, but the FcR binding is actually different for each monomer. For example, the CH2D may be directed to EGFR and the FcR on one monomer component may be selective for FcgRIII and the second monomer of the dimer also targeted to EGFR but the FcR binding eliminated in favor of complement binding or binding to FcRIIb.

The CH2D multimer may comprise a third, fourth, fifth, and/or sixth CH2 domain. In some embodiments, the third immunoglobulin CH2 domain is specific for a target for which the first immunoglobulin CH2 domain is specific. The third immunoglobulin CH2 domain may be specific for a target for which the second immunoglobulin CH2 domain is specific, or for a target for which both the first and second immunoglobulin CH2 domain is specific. In some embodiments, the third immunoglobulin CH2 domain is specific for a third target for which neither the first immunoglobulin CH2 domain nor the second immunoglobulin CH2 domain is specific.

In some embodiments, the fourth immunoglobulin CH2 domain is specific for a target for which the first immunoglobulin CH2 domain is specific, and/or a target for which the second immunoglobulin CH2 domain is specific, and/or for a target for which the third immunoglobulin CH2 domain is specific. In some embodiments, the fourth immunoglobulin CH2 domain is specific for a fourth target for which neither the first immunoglobulin CH2 domain, the second immunoglobulin CH2 domain, nor the third immunoglobulin CH2 domain is specific.

In some embodiments, the fifth immunoglobulin CH2 domain is specific for a target for which the first immunoglobulin CH2 domain is specific, and/or a target for which the second immunoglobulin CH2 domain is specific, and/or for a target for which the third immunoglobulin CH2 domain is specific, and/or for a target for which the fourth immunoglobulin CH2 domain is specific. In some embodiments, the fifth immunoglobulin CH2 domain is specific for a fifth target for which neither the first immunoglobulin CH2 domain, the second immunoglobulin CH2 domain, the third immunoglobulin CH2 domain, nor the fourth immunoglobulin domain is specific.

In some embodiments, the sixth immunoglobulin CH2 domain is specific for a target for which the first immunoglobulin CH2 domain is specific, and/or a target for which the second immunoglobulin CH2 domain is specific, and/or for a target for which the third immunoglobulin CH2 domain is specific, and/or for a target for which the fourth immunoglobulin CH2 domain is specific, and/or for a target for which the fifth immunoglobulin CH2 domain is specific. In some embodiments, the sixth immunoglobulin CH2 domain is specific for a sixth target for which neither the first immunoglobulin CH2 domain, the second immunoglobulin CH2 domain, the third immunoglobulin CH2 domain, the fourth immunoglobulin domain, nor the fifth immunoglobulin domain is specific.

### Serum Half-Life and Effector Molecule Binding

Serum half-life of an immunoglobulin is mediated by the binding of the F_{c} region to the neonatal receptor FcRn. The alpha domain is the portion of FcRn that interacts with the CH2 domain (and possibly CH3 domain) of IgG, and possibly with IgA, and IgD or with the CH3 domain (and possibly CH4 domain) of IgM and IgE. Several studies support a correlation between the affinity for FcRn binding and the serum half-life of an immunoglobulin.

In some embodiments, the CH2D multimer has a greater half-life in a media (e.g., serum) as compared to the half life of a CH2D monomer derived from the CH2D multimer. Although the native IgG molecule comprises two FcRn binding sites, it is unknown whether these may simultaneously engage two FcRn receptors on the surface of a cell. The relative orientation of the CH2 domains in the whole or Fc fragment of an immunoglobulin is tightly constrained by the covalent linkage of the hinge region at one end and the tight non-covalent interaction between the two CH3 domains of IgG at the other. Freeing the CH2 domains from one or both such constraints, as in the case of the various illustrated CH2D multimers, may potentially enhance FcRn interaction by avidity.

Modifications may be made to the CH2D to modify (e.g., increase or decrease) the affinity and/or avidity the immunoglobulin has for FcRn (see, for example, U.S. Patent Application No. 2007/0135620). Modifications may include mutations (amino acid substitutions, deletions, physical modifications to amino acids) of one or more amino acid residues in one or more of the CH2 domains. Modifications may also include insertion of one or more amino acid residues or one or more binding sites (e.g., insertion of additional binding sites for FcRn). A modification may, for example, increase the affinity for FcRn at a lower pH (or higher pH). The present invention is not limited to the aforementioned modifications.

In some embodiments, the CH2D multimer comprises at least one binding site for FcRn (e.g., wild type, modified, etc.). In some embodiments, the CH2D multimer comprises at least two binding sites for FcRn (e.g., wild type, modified, etc.). In some embodiments, the multimer comprises three or more binding sites for FcRn. None, one, or more of the binding sites for FcRn may be modified (e.g. example mutated).

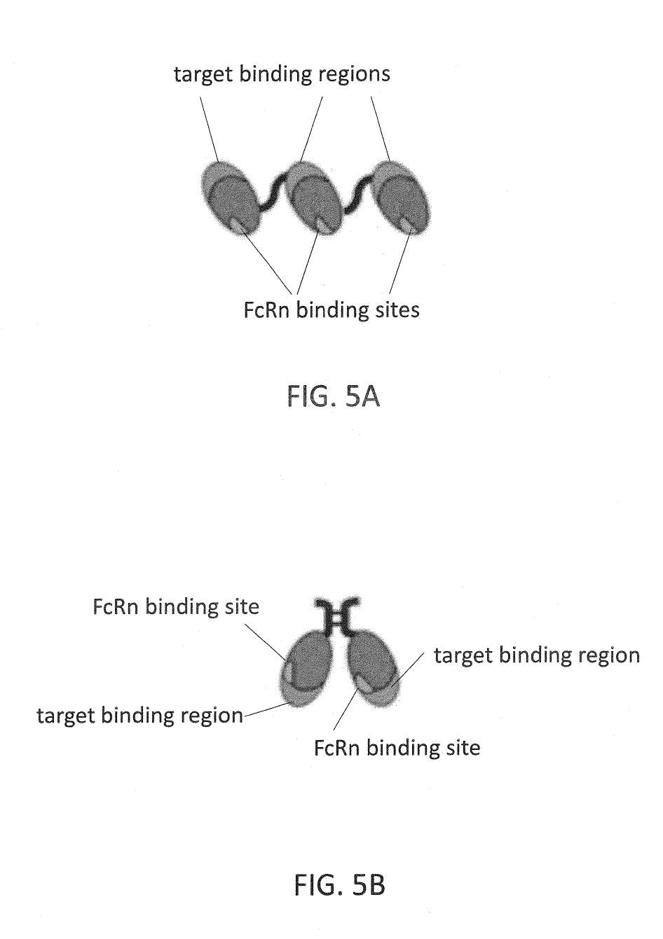

FIG. 5A illustrates an example of a multimer comprising three CH2 domains. Each CH2 domain comprises an FcRn receptor binding site (e.g., unmodified or modified). FIG. 5B illustrates an example wherein a first CH2 domain is linked to a second CH2 domain via a hinge component. Both CH2 domains comprise a FcRn receptor. Alternatively, in some embodiments, none of the CH2 domains comprise a FcRn (or a functional FcRn) binding site.

F_{c} receptors are receptors found on certain immune system cells, for example phagocytes (e.g., macrophages), natural killer cells, neutrophils, and mast cells. F_{c} receptor activation can cause phagocytic or cytotoxic cells to destroy the target antigen bound to the antibody's paratope. F_{c} receptors are classified based on the isotype of antibody they recognize. For example, F_{c}γ receptors bind IgG, F_{c}α receptors bind IgA, F_{c}δ receptors bind IgD, F_{c}ε receptors bind IgE, and F_{c}μ receptors bind IgM. While all of the aforementioned F_{c} receptors (excluding FcRn) are involved in immune responses, a subset of the F_{c}γ receptors is considered to be the most potent pro-inflammatory receptors. In the case of F_{c}γ receptors, receptor activation leads to activation of signalling cascades via motifs, for example an immunoreceptor tyrosine-based activation motif (ITAM), which causes activation of various other kinase reaction cascades depending on the cell type. Certain Fc□ receptors antagonize the signalling of the pro-inflammatory Fc□ receptors, and these anti-inflammatory receptors typically are linked to immunoreceptor tyrosine-based inhibition motif (ITIM) (see, for example Ravetch et al., (2000) Science 290:84-89).

Without wishing to limit the present invention to any theory or mechanism, it is believed that the CH2 domains of IgG, IgA, and IgD (or the equivalent CH3 domain of IgM and IgE) are responsible for all or most of the interaction with F_{c} receptors (e.g., F_{c}γ, F_{c}α, F_{c}δ, F_{c}ε, F_{c}μ). In some embodiments, it may be useful to limit the ability of the multimeric CH2Ds to functionally bind F_{c} receptors (e.g., pro-inflammatory F_{c}γ, F_{c}α, F_{c}δ, F_{c}ε, F_{c}μ), for example to help prevent adverse immune response effects. In such cases, retaining only one functional binding interaction with a particular pro-inflammatory F_{c} receptor will confer properties most analogous to those of a native immunoglobulin. In contrast, in some embodiments it may be useful to enhance the ability of the multimeric CH2D to functionally bind F_{c} receptors (Fey, F_{c}α, F_{c}δ, F_{c}ε, F_{c}μ), for example if one wishes to perform research experiments to study F_{c} receptors. In another example, one may target a specific Fc receptor to either agonize or antagonize that receptor. Such modifications of the CH2D to allow for specific Fc receptor interactions are contemplated herein.

As discussed above in the context of FcRn binding, the naturally occurring CH2 domains in the F_{c} portion of an antibody intrinsically possess a dimeric configuration, presenting two potential F_{c} receptor binding sites. However, it is not certain that both CH2 domains within a single IgG molecule can simultaneously bind to two F_{c} receptors located on the same cell surface. The hinge region restricts the N-termini of the CH2 domains, while the C-termini are constrained by the linkage to the CH3 domains, so that there are limited conformations of the CH2 domains within the immunoglobulin. Freeing the CH2 domains of one or both of these constraints may result in avidity effects that increase the binding of certain FcγR receptors. Furthermore, the pro-inflammatory receptors in particular appear to be triggered to signal by clustering of these relatively low affinity receptors. Such clustering is usually caused by the F_{c} portions of multiple IgG molecules where the Fab arms are bound to an array of antigen on a virus or a bacterial cell surface. Thus, a pro-inflammatory response is triggered only when multiple IgG molecules are bound to an array of the corresponding antigen, limiting the inflammation to an area where the invading pathogen is located. The high serum concentration of the IgG does not trigger pro-inflammatory signalling because of the low affinity and absence of any avidity effects in serum. It is possible that two or more CH2 domains that are not constrained by the normal IgG context may be able to trigger directly an inflammatory response, which would be systemic and highly undesirable to many therapeutic interventions. CH2D multimers that retain only one domain that can activate a pro-inflammatory response may be the most effective for treatments, potentially behaving most like a native IgG in terms of FcR signalling.

In some embodiments, the multimeric CH2D comprises no more than one functional binding site able to activate pro-inflammatory FcγR. In some embodiments, only one immunoglobulin CH2 domain has a functional F_{c} receptor-binding region for binding to a target F_{c} receptor to effectively activate an immune response. Other F_{c} receptor-binding regions (in other CH2 domains) may be non-functional F_{c} receptor-binding regions or F_{c} receptor-binding regions or may be substantially absent (e.g., deleted) from the CH2 domain. In some embodiments, the term “functional F_{c} receptor-binding region” refers to the ability of the binding of the F_{c} receptor-binding region to the F_{c} receptor to cause activation of a signalling cascade, for example via an ITAM. In some embodiments, a “non-functional F_{c} receptor-binding region” may refer to an F_{c} receptor-binding region that cannot bind to the F_{c} receptor (or cannot completely bind), or to a F_{c} receptor-binding region that can bind to the F_{c} receptor but cannot cause activation of a signalling cascade (e.g., via an ITAM).

In some embodiments, at least one of the immunoglobulin CH2 domains does not have a functional F_{c} receptor-binding region for binding to a target F_{c} receptor to effectively activate an immune response. In some embodiments, the multimer CH2D lacks entirely a functional F_{c} receptor-binding region for binding to a target F_{c} receptor to effectively activate an immune response.

The CH2 domains of IgG, IgA, and IgD (or the equivalent CH3 domain of IgM and IgE) also have binding sites for complement. In some embodiments, it may be useful to limit the ability of the multimer to activate a complement cascade, for example to help prevent adverse immune response effects for reasons analogous to those discussed above in relation to pro-inflammatory F_{c} receptor binding. In contrast, in some embodiments it may be useful to enhance the ability of the multimer CH2D to activate a complement cascade, for example if one wishes to perform research experiments to study complement or in anti-cancer applications.

In some embodiments, the multimeric CH2D comprises no more than one functional binding site for complement. In some embodiments, only one immunoglobulin CH2 domain has a functional binding site for a complement molecule (functional referring to the ability of the binding site to initiate a complement cascade). In some embodiments, at least one CH2 domain of the multimer does not have a functional binding site for a complement molecule. In some embodiments, at least one of the immunoglobulin CH2 domains (e.g., a complement binding site) is modified (e.g., mutated, etc.) so as to reduce or eliminate complement activation. Or, the complement binding site may be selected from an immunoglobulin isotype having reduced or absent ability to activate a complement cascade.

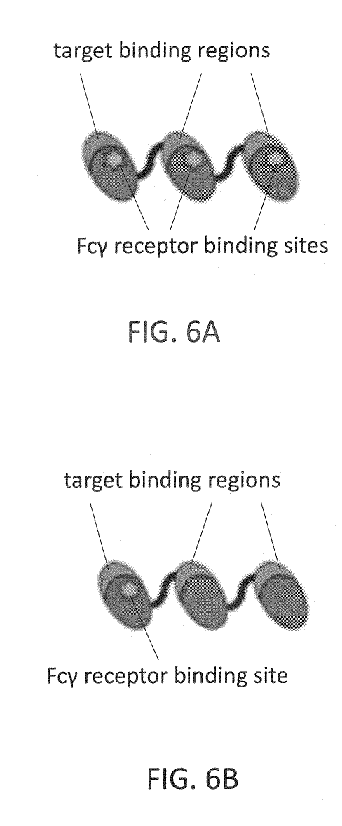

FIG. 6A illustrates an example of a CH2D multimer comprising three CH2 domains. Each CH2 domain comprises a F_{c}γ receptor binding site (e.g., unmodified or modified). FIG. 6B illustrates an example wherein only one CH2 domain comprises a F_{c}γ receptor binding site (e.g., unmodified or modified).

### Stability

Stability is an important property of a protein, and it can determine the ability of the protein to withstand storage or transport conditions as well as affect the protein's half-life after administration (e.g., in serum). In some embodiments, the CH2Ds are contained in a pharmaceutical composition for providing increased stability. Pharmaceutical compositions for antibodies and peptides are well known to one of ordinary skill in the art. For example, U.S. Pat. No. 7,648,702 features an aqueous pharmaceutical composition suitable for long-term storage of polypeptides containing an Fc domain of an immunoglobulin. Pharmaceutical compositions may comprise buffers (e.g., sodium phosphate, histidine, potassium phosphate, sodium citrate, potassium citrate, maleic acid, ammonium acetate, tris-(hydroxymethyl)-aminomethane (tris), acetate, diethanolamine, etc.), amino acids (e.g., arginine, cysteine, histidine, glycine, serine, lysine, alanine, glutamic acid, proline), sodium chloride, potassium chloride, sodium citrate, sucrose, glucose, mannitol, lactose, glycerol, xylitol, sorbitol, maltose, inositol, trehalose, bovine serum albumin (BSA), albumin (e.g., human serum albumin, recombinant albumin), dextran, PVA, hydroxypropyl methylcellulose (HPMC), polyethyleneimine, gelatin, polyvinylpyrrolidone (PVP), hydroxyethylcellulose (HEC), polyethylene glycol (PEG), ethylene glycol, dimethylsulfoxide (DMSO), dimethylformamide (DMF), hydrochloride, sacrosine, gamma-aminobutyric acid, Tween-20, Tween-80, sodium dodecyl sulfate (SDS), polysorbate, polyoxyethylene copolymer, sodium acetate, ammonium sulfate, magnesium sulfate, sodium sulfate, trimethylamine N-oxide, betaine, zinc ions, copper ions, calcium ions, manganese ions, magnesium ions, CHAPS, sucrose monolaurate, 2-O-beta-mannoglycerate, the like, or a combination thereof. The present invention is in no way limited to the pharmaceutical composition components disclosed herein, for example pharmaceutical compositions may comprise propellants (e.g., hydrofluoroalkane (HFA)) for aerosol delivery. U.S. Pat. No. 5,192,743 describes a formulation that when reconstituted forms a gel which can improve stability of a protein of interest (e.g., for storage). Pharmaceutical compositions may be appropriately constructed for some or all routes of administration, for example topical administration (including inhalation and nasal administration), oral or enteral administration, intravenous or parenteral administration, transdermal administration, epidural administration, and/or the like. For example, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In some embodiments, the multimer CH2Ds are bound to a scaffold that confers increased stability (e.g., serum half-life). Dextrans and various polyethylene glycols (PEG) are extremely common scaffolds for this purpose (see, for example, Dennis et al., 2002, Journal of Biological Chemistry 33:238390). The scaffolds may be bound by a variety of mechanisms, for example via chemical treatments and/or modification of the protein structure, sequence, etc. (see, for example, Ashkenazi et al., 1997, Current Opinions in Immunology 9:195-200; U.S. Pat. No. 5,612,034; U.S. Pat. No. 6,103,233). The scaffold (e.g., dextran, PEG, etc.) may be bound to the CH2D through a reactive sufhydryl by incorporating a cysteine at the end of the protein opposite the binding loops. Such techniques are well known in the art. In another example, one of the CH2Ds of a trimer may bind specifically to albumin to utilize the albumin in serum to increase circulating half-life.

Choosing pharmaceutical compositions that confer increased protein stability or binding of the peptides (e.g., CH2Ds) to scaffolds that confer increased protein stability are not the only ways in which the stability of the protein can be improved. In some embodiments, the multimer CH2Ds of the present invention may be modified to alter their stability. Again, the term “modified” or “modification,” can include one or more mutations, deletions, substitutions, physical alteration (e.g., cross-linking modification, covalent bonding of a component, post-translational modification, e.g., acetylation, glycosylation, the like, or a combination thereof), the like, or a combination thereof. Gong et al. (2009, Journal of Biological Chemistry 284:14203-14210) shows examples of modified CH2 domains having increased stability. For example, human γ1 CH2 was cloned and a variety of cysteine mutants were created. The stability of the mutants with respect to the wild type CH2 was determined (e.g., the proteins were subjected to high temperatures and urea treatment). One mutant (m01, which comprised additional disulfide bonds) was particularly stable having a higher melting temperature, increased resistance to urea-induced unfolding, and increased solubility. Mutants such as these may be particularly useful for constructing multimers according to the present invention. Multimers with higher melting temperatures and/or increased resistance to urea-induced unfolding and/or and increased solubility may be more likely to withstand storage and transport conditions as well as have increased serum stability after administration.

Due to the unstable nature of proteins, pharmaceutical compositions are often transported and stored via cold chains, which are temperature-controlled uninterrupted supply chains. For example, some pharmaceutical compositions may be stored and transported at a temperature between about 2 to 8 degrees Celsius. Cold chains dramatically increase the costs of such pharmaceutical compositions. Without wishing to limit the present invention to any theory or mechanism, it is believed that increasing the stability of the multimers of the present invention (e.g., via modification, via pharmaceutical compositions) may help reduce or eliminate the need to store and transport the multimers via cold chains.

The aforementioned pharmaceutical compositions and protein modifications to increase protein stability can be applied to monomeric antibody domains such as those described in U.S. Patent Application 2009/032692.

### Linkers

Linkers may be used to link two or more CH2 domains together, for example the first CH2 domain and the second CH2 domain may be linked via a linker. Linkers may affect the positioning of the CH2 domains, the accessibility of functional regions of the CH2 domains, and the overall structure of the multimeric proteins. For example, proline residues are known to bend or kink the structure of a protein, and thus a linker comprising one more proline residues may bend or kink the structure of the CH2D multimer. Structure of the multimer or portions thereof can in some cases affect the ability of the multimer to perform certain functions, for example binding to target antigens, binding to Fc receptors (including FcRn receptors), binding to cascade molecules, and the like.

A linker, for example, may include but is not limited to a peptide of various amino acid lengths and/or sequences. In some embodiments, the linker is between about 5 to 10 amino acids in length. In some embodiments the linker is between about 10 to 15 amino acids in length. In some embodiments, the linker is between about 15 to 20 amino acids in length, or more than about 20 amino acids in length. The linker may be encoded for in the gene which encodes for the multimer Ch2Ds, or the linker may be covalently bonded (e.g., cross-linked) to a portion of the CH2D multimer.

The linkers may be covalent or very tight non-covalent linkages; chemical conjugation or direct gene fusions of various amino acid sequences, e.g., those (a) rich in Glycine Serine, Proline, Alanine, or (b) variants of naturally occurring linking amino acid sequences that connect immunoglobulin domains. Typical lengths may range from 5 up to 20 or more amino acids, however the present invention is not limited to this length. The optimal lengths may vary to match the spacing and orientation of the specific target antigen(s), minimizing entropy but allowing effective binding of multiple antigens. Various arrangements are given in the figures.

In some embodiments, the linker functions as a multimerizing (e.g., dimerizing, trimerizing, etc.) domain or comprises a multimerizing domain. The length and composition of the linker may be used to modulate the binding of a dimeric CH2 domain to a multimeric antigen, the spacing and orientation of the antigen being matched by composition and length of the linker. Variants of leucine zipper domains may be used to homo-dimerize or hetero-dimerize (e.g. myc-max) CH2 domains. Isoleucine zippers (e.g. GCN4) can be used to direct trimerization, with disulphide linking incorporated at one end of the domain. In some embodiments, the linker comprises a non-peptide component (e.g., a sugar residue, a heavy metal ion, a chemical agent such as a therapeutic chemical agent, etc.). Linkers and/or multimerizing domains may be attached to the N-terminus (or the N-terminus region), or the C-terminus (or the C-terminus region), or any other region of the CH2 domain. The linker is not limited to these attachment means, configurations, and/or functions.

Referring now to FIG. 1A, a target binding region (e.g., CDR domain or functional fragment thereof) of a first CH2 domain (left) is linked via a linker to a second CH2 domain (right). FIG. 1B shows a different configuration wherein the first CH2 domain (left) is linked via a linker to a target binding region of a second CH2 domain (right). FIG. 1C shows five CH2 domains, wherein a first CH2 domain being linked via a linker to a target binding region of a second CH2 domain, a different region of the second CH2 domain is linked via a linker to a third CH2 domain, a different region of the third CH2 domain is linked via a linker to a fourth CH2 domain, and a different region of the fourth CH2 domain is linked via a linker to a fifth CH2 domain.

Referring now to FIG. 2, linkers may comprise one or more multimerizing domains. In some embodiments, two or more CH2 domains are linked via the multimerizing domains of the linkers. FIG. 2A shows two CH2 domains, each comprising a linker having a multimerizing domain. The two CH2 domains are linked via the bonding of the multimerizing domains. FIG. 2B shows three CH2 domains, each comprising a linker having a multimerizing domain, and the three CH2 domains are linked together via the bonding of the multimerizing domains. FIG. 2C shows four CH2 domains, each comprising a linker having a multimerizing domain, and the four CH2 domains are linked together via the bonding of the multimerizing domains.

FIG. 2D shows four CH2 domains: a first CH2 domain (left), a second CH2 domain (middle-left), a third CH2 domain (middle-right), and a fourth CH2 domain (right). The first and second CH2 domains are linked via a linker (e.g., the first CH2 domain is linked via a linker to a target binding region of the second CH2 domain), and the third and fourth CH2 domains are linked via a linker (e.g., the fourth CH2 domain is linked via a linker to a target binding region of the third CH2 domain). The second CH2 domain and third CH2 domain further comprise an additional linker, each additional linker comprising a multimerizing domain. The first and second CH2 domains are connected to the third and fourth CH2 domains via bonding of the multimerizing domains.

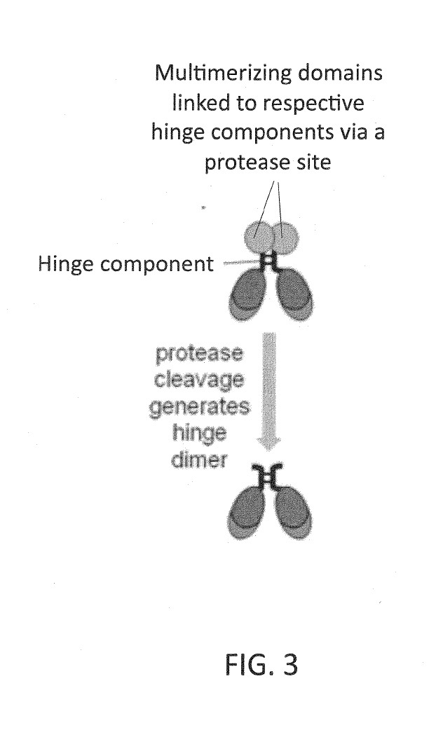

Referring now to FIG. 3, in some embodiments, CH2 domains may be linked via hinge components. For example, a first CH2 domain may comprise a first half hinge component which is capable of binding a second half hinge component of a second CH2 domain. In some embodiments, the hinge components may comprise one or more multimerizing domains. The multimerizing domains may be configured such that they can be cleaved subsequently from the hinge components via proteolysis. Any protease might be used that exhibits sufficient specificity for its particular recognition sequence designed into the linker, but does not cleave any other sequence in the CH2D molecule. The cleavage preferably occurs at the extreme end of the recognition motif, so that no additional amino acid residues that are part of the recognition site are retained by the final CH2D molecule. The protease should ideally be a human enzyme that would have little effect on a patient if trace amounts were carried over following purification. Blood clotting factors such as Factor X or thrombin might be particularly useful in removing multimerization domains

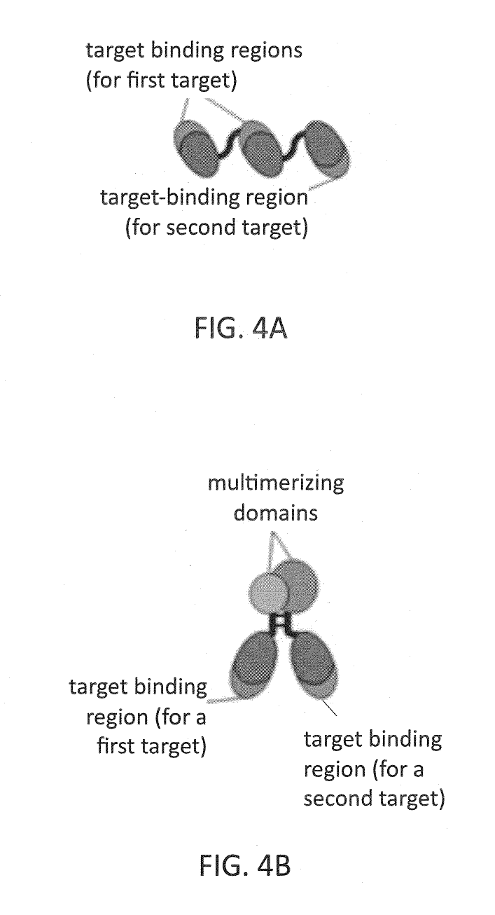

Referring now to FIG. 4, as previously discussed, the multimeric CH2D may be specific for one or more target antigens. For example, the first CH2 domain may be specific for a first target, and the second CH2 domain may be specific for the first target or for a second target. FIG. 4A illustrates a multimer comprising a first CH2 domain (left), a second CH2 domain (middle), and a third CH2 domain (right). The first CH2 domain and the second CH2 domain each comprise a target binding region specific for a first target, while the third CH2 domain comprises a target binding region specific for a second (different) target. FIG. 4B illustrates a multimer comprising a first CH2 domain (left) and a second CH2 domain (right). The first CH2 domain has a target binding region specific for a first target and the second CH2 domain has a target binding region specific for a second target. The two CH2 domains are linked via hinge components, each comprising a multimerizing domain.

In some embodiments, the N-terminus of the first immunoglobulin CH2 domain is linked to the C-terminus of the second immunoglobulin CH2 domain. In some embodiments, N-terminus of the second immunoglobulin CH2 domain is linked to the C-terminus of the first immunoglobulin CH2 domain. In some embodiments, the C-terminus of the first immunoglobulin CH2 domain is linked to the C-terminus of the second immunoglobulin CH2 domain. In some embodiments, the N-terminus of the first immunoglobulin CH2 domain is linked to the N-terminus of the second immunoglobulin CH2 domain.

### Methods

The multimeric CH2Ds may be important tools for treating or managing diseases or conditions. The present invention also features methods of treating or managing a disease condition using the CH2Ds of the present invention. The method may comprise obtaining CH2D multimers (e.g., comprising a first immunoglobulin CH2 domain linked to a second immunoglobulin CH2 domain, e.g., via a linker) specific for a first target related to the disease or condition and introducing the CH2Ds into a mammal, e.g., patient, (e.g., to a tissue of the mammal). The CH2D multimers, being specific for the first target, may bind to the first target. Binding may function to cause the neutralization or destruction of the target. The target may be, for example, a cell, a tumor cell, an immune cell, a protein, a peptide, a molecule, a bacterium, a virus, a protist, a fungus, the like, or a combination thereof. For example, destruction of a target cell (in this example a tumor) could be achieved by therapy using the following CH2D as API: a first CH2D directed to a particular tumor surface antigen (such as an EGFR, IGFR, nucleolin, ROR1, CD20, CD19, CD22, CD79a, stem cell markers) is linked to a second CH2D that binds to a different tumor surface antigen on the same cell from that bound by the first domain. This arrangement can enhance the specificity of the CH2D dimer for the tumor over any normal tissues since it will bind more tightly to cells displaying both of the two antigens. The dimer described above is further linked to an additional CH2D (now a trimer) that binds to an immune effector cell surface antigen (for example, a T-cell specific antigen like CD3, or an NK cell specific surface antigen, like Fc-gamma-RIIIa). In this way, the specific binding to the tumor by the two targeting domains leads to recruitment of a T-cell (or of an NK cell) that destroys the tumor cell.

In some embodiments, the CH2Ds comprise an agent that functions to neutralize or destroy the target. Agents may include but are not limited to a peptide, a chemical, a toxin, and/or the like. In some embodiments, the agent is inert or has reduced activity when linked to the CH2D; however, the agent may be activated or released upon uptake or recycling or enzymatic cleavage in a diseased tissue.

Because of the ability of the multimeric CH2Ds of the present invention to bind to various targets, the CH2D may be used for detection of diseases and/or conditions. For example, a method of detecting a disease or condition (e.g., in a mammal) may comprise obtaining a CH2D multimer (e.g., comprising a first immunoglobulin CH2 domain linked□ to a second immunoglobulin CH2 domain) and introducing the CH2D multimer into a sample (e.g., sample derived from the mammal). In some embodiments, the CH2D multimer binds to a target in the sample and has a specific label conjugated to the CH2D. The target is associated with the disease or condition.

Various methods may be used for detecting the binding of the CH2D multimer to the target in the sample. Such methods are well known to one of ordinary skill in the art. In some embodiments, detecting binding of the CH2D multimer to the target indicate the presence of the disease or condition in the sample.

Methods for screening protein specificity are well known to one of ordinary skill in the art. The present invention also features methods of identifying a CH2D multimer that specifically binds a target. The method may comprise obtaining a library of particles which display on their surface a CH2D or CH2D multimer of the present invention (e.g., a CH2D multimer comprising a first immunoglobulin CH2 domain linked to a second immunoglobulin CH2 domain) and introducing the target to the library of particles. Particles from the library that specifically bind to the target can be selected via standard methods well known to one of ordinary skill in the art. CH2D scaffolds may provide a means of obtaining a greater diversity of loops to discover those that have an increased probability of binding a target compared to the diversity of loops that might be available in a whole antibody or variable region-containing format (see, for example, Xiao et al., 2009, Biological and Biophysical Research Communications 387:387-392).

Alternatively, libraries of displayed monomeric CH2D variants may be used to first isolate CH2 domains that specifically bind to individual target antigens. The variants that bind can then be combined to form multimers with specificity for one or more target antigens. Libraries of multimeric CH2Ds may be constructed that are based on two CH2Ds that were previously isolated from monomeric CH2D libraries. Such libraries can be used to optimize the length and/or sequence of the linker to maximize binding.

### EXAMPLES

### Methods and Data for Generation and Characterization of CH2D Monomers and Dimers

The stability of native single domain isolated CH2 (CH2D), engineered CH2 (m01) and a dimer of the native CH2 (dimer CH2) protein were assessed in cynomolgus monkey serum (FIG. 7). Serum was incubated for 7 days at 37° C., and samples of serum were collected each day. Serum proteins in each daily sample were subjected to gel electrophoresis followed by Western blotting to assess the presence of intact CH2D over the 7 day timecourses. Mouse anti-HIS monoclonal antibody and alkaline phosphatase conjugated goat-anti-mouse IgG were used as primary and secondary antibodies, respectively. CH2D was detected in the samples at a similar concentration over the 7 days at 37° C. (FIG. 7, right panel). The stability of CH2D dimer was similar to that of the CH2D monomer (FIG. 7, right panel vs. left panel). A short stabilized mutant monomer of CH2D (m01) (Gong et al. (2009) Journal of Biological Chemistry 84(21):14203-14210), which has a leucine to cysteine substitution at amino acid 12 and a lysine to cysteine substitution at amino acid 104 (FIG. 8A), was also stable for 7 days at 37° C. (FIG. 7, middle panel). These data confirm that CH2D monomers and dimers are stable and not significantly degraded or metabolized in non-human primate serum, validating their potential use as human therapeutic agents.

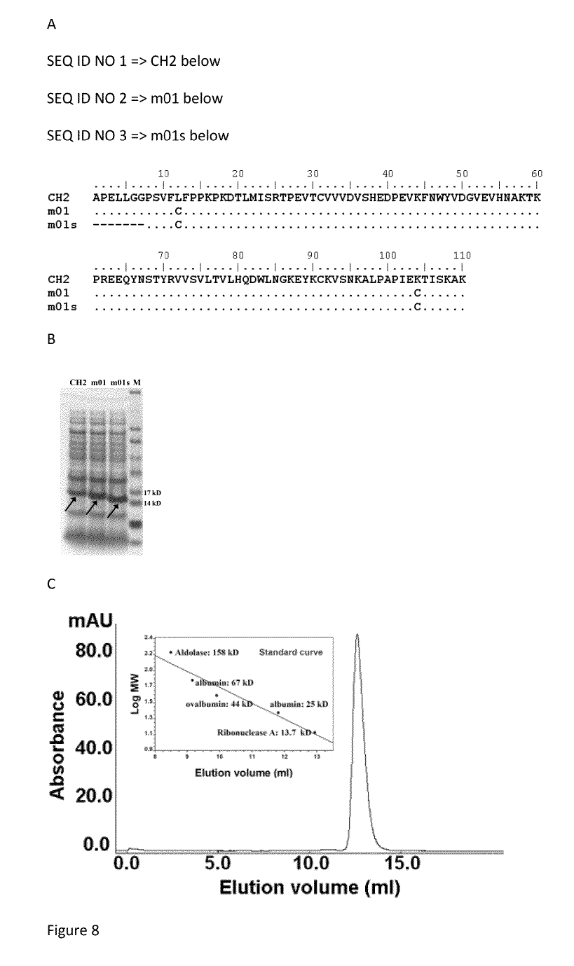

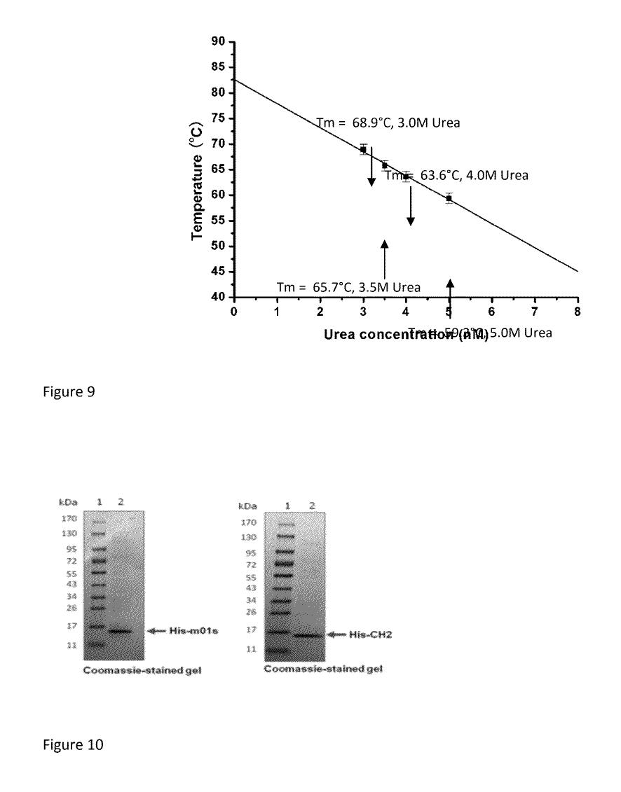

The mutant CH2D monomer m01 (described in the stability section of the provisional application and FIG. 8A) was engineered to generate a shorter version termed m01s. The first seven residues were removed from m01 to make m01s (FIG. 8A). The expression of soluble m01s by the transformed E. coli was higher than that of wild-type CH2 and m01 (FIG. 8B). In addition, m01s exists as a monomer in PBS at pH 7.4 based on size exclusion chromatography analysis (FIG. 8C). The Tm of m01 and m01s were calculated and compared using Circular Dichroism. The thermo-induced unfolding of the proteins was measured in the presence of 3 M, 3.5 M, 4 M, and 5 M Urea in PBS at pH 7.4. The Tm value of m01 was 73.8° C., and the Tm value of m01s was 82.6° C. (FIG. 9). Therefore, m01s is a more stable protein (Gong et al., unpublished).

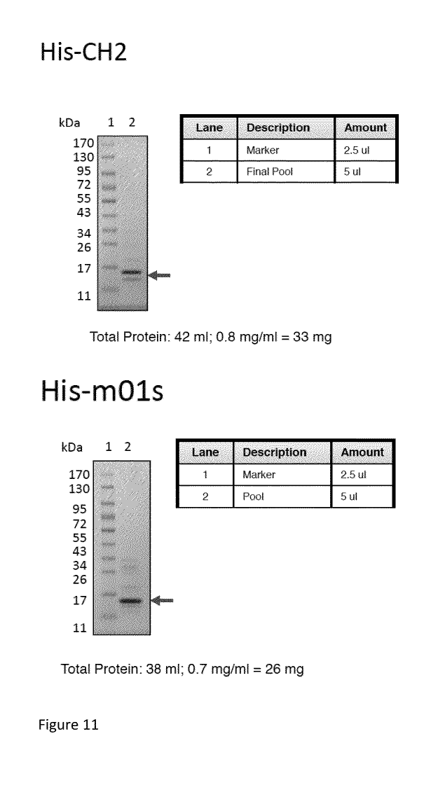

To determine the best conformation of CH2D for optimal binding to its target molecule(s), multiple CH2D variants were produced in and purified from E. coli using methods established by the Dimitrov laboratory (Gong et al. (2009) Journal of Biological Chemistry 84(21): 4203-14210). CH2D constructs were also commercially produced and purified by Blue Sky BioServices using the same production and purification methods. Blue Sky BioServices initiates their purification of a new protein using 1 L preparations before they scale-up the process. Their scale-up utilizes 10 L batches, and any endotoxins are removed. Optimizing these production and purification processes will facilitate the development of a strain suitable for commercialization. FIG. 10 demonstrates the abundance of wild-type CH2D and mutant CH2D (m01s) protein isolated from 1 L preparations of E. coli engineered to produce the heterologous proteins. Blue Sky BioServices was able to produce 33 mg of CH2D dimer (FIG. 11, upper panel) and 26 mg of the stable CH2D monomer (m01s) (FIG. 11, lower panel) using their large-scale production methods, demonstrating the potential for commercialization of the CH2D production process.

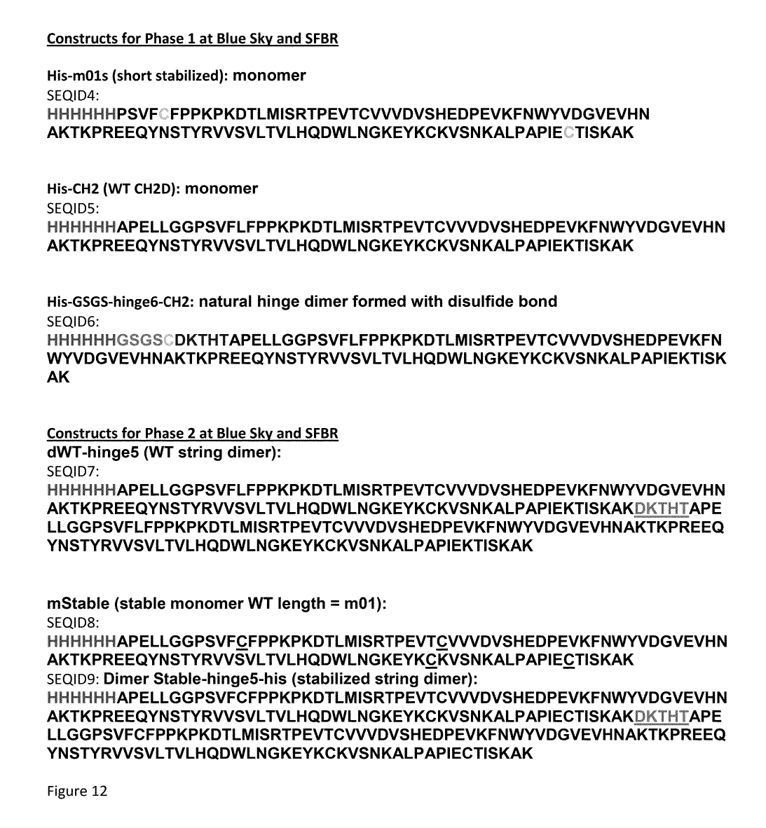

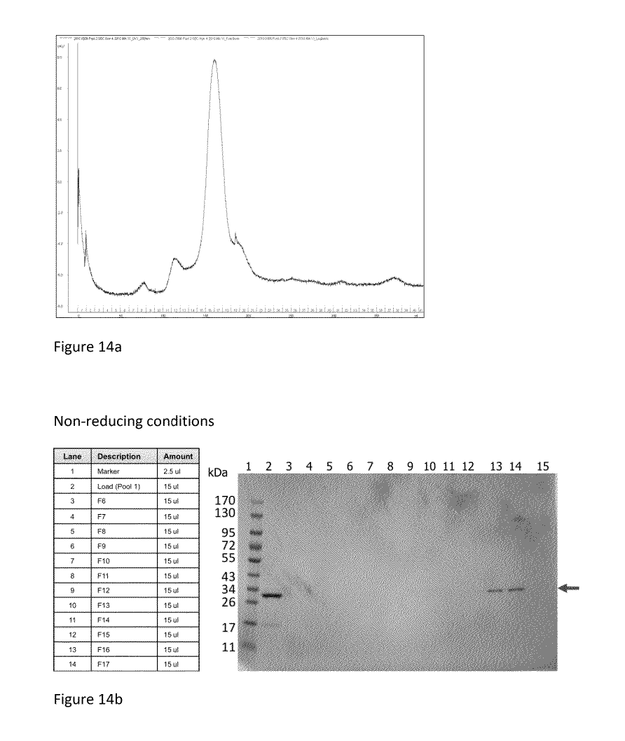

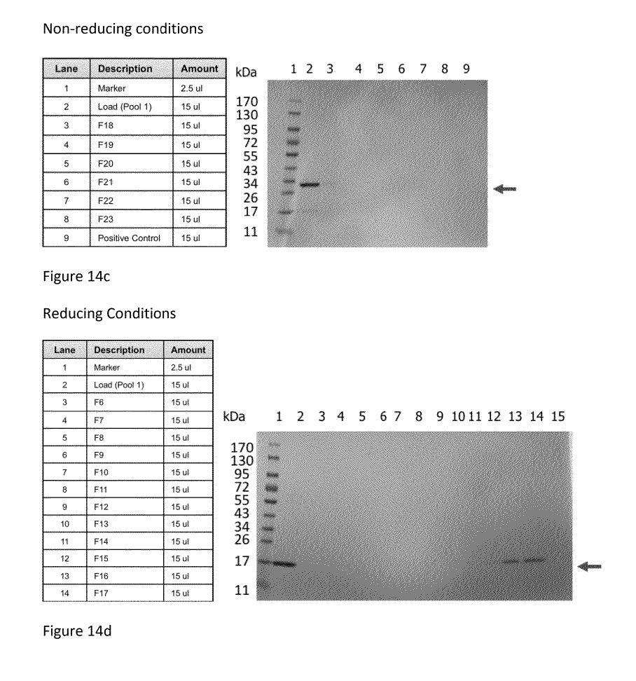

In order to identify the optimal conformation for CH2D to maximize its binding and effector functions, multiple variants of CH2D dimers were generated (Table 1); the corresponding sequences for each construct are provided in FIG. 12. The yield of these constructs varied using the previously described E. coli production and purification methods (Table 1). CH2D constructs with an additional cysteine and hinge region from IgG at the N or C-terminal (natural hinge) were assessed (FIG. 13). To increase the stability and binding of CH2D to its target molecules, the HIS-TAGs were moved from the COOH-terminus to the NH2-terminus to avoid interference with the binding of FcRn, and a GSGS spacer was added between the cysteine and His-TAG (FIG. 13). A FLAG tag was added to the carboxy terminus of the CH2-IgG1hinge5-cysteine-His6 construct in order to be able to detect the expression of this CH2D using FLAG-specific antibodies (FIG. 13, first construct). Blue Sky BioServices successfully purified His-GSGS-hinge6-CH2 (FIG. 12) using size exclusion chromatography followed by SDS-PAGE under both reducing and non-reducing conditions. There were 10 mL fractions collected during the size exclusion chromatography (FIG. 14, upper panel), and each fraction was separately evaluated using SDS-PAGE under both non-reducing (FIGS. 14B and 14C) and reducing conditions (FIGS. 14D and 14E). There was a specific and distinct band corresponding to the expression of His-GSGS-hinge6-CH2 (indicated by arrow). These data demonstrate the ability to obtain high-purity preparations of CH2D dimers, which will be required for future therapeutic applications.

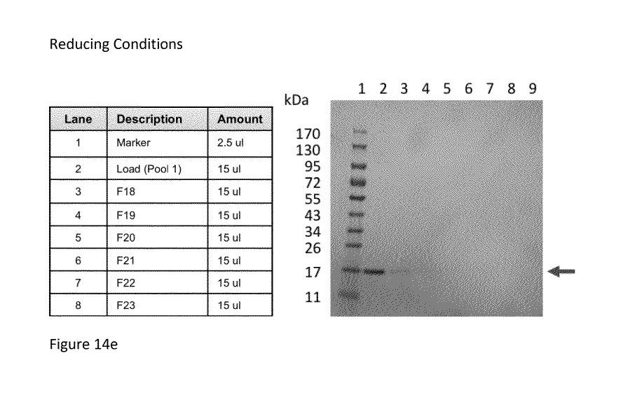

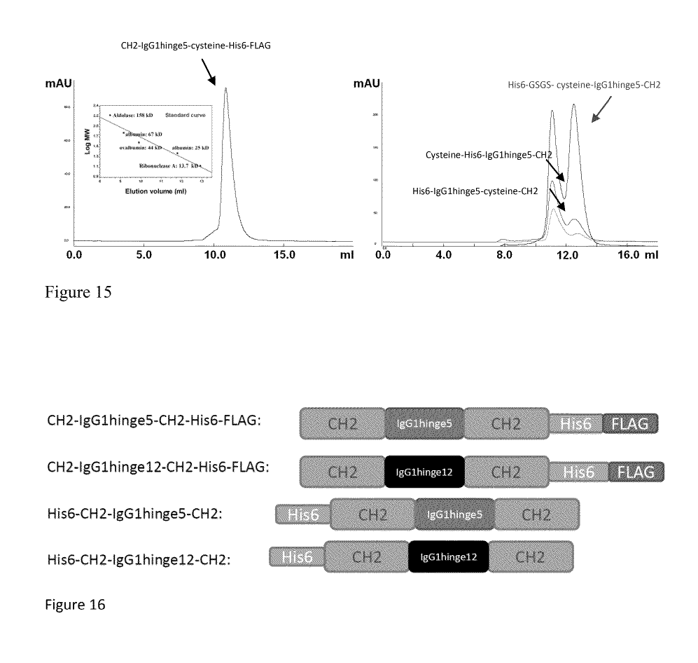

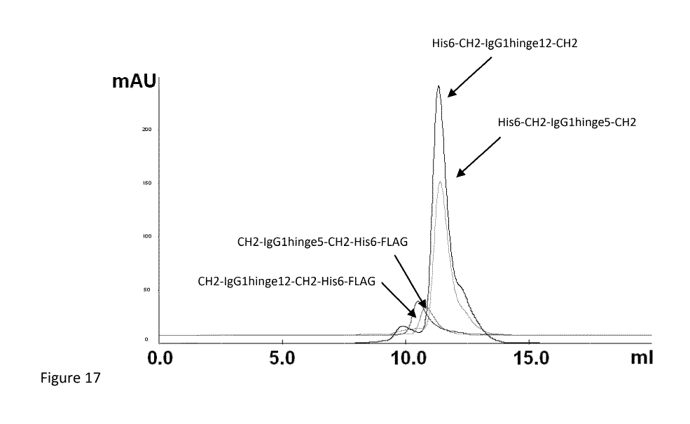

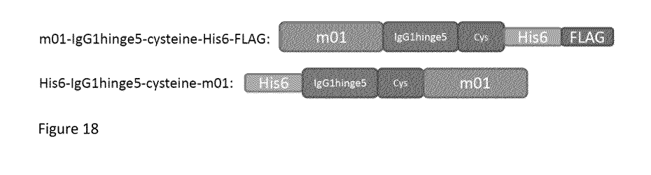

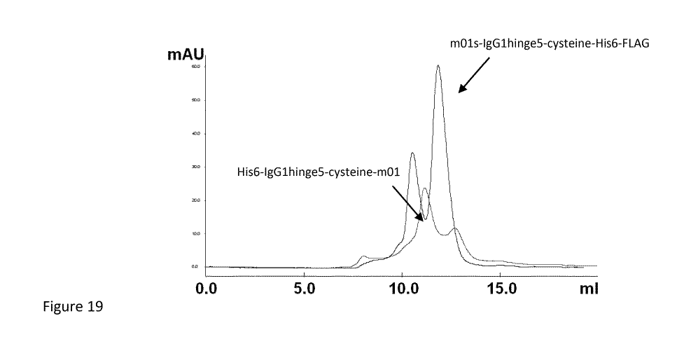

The ability of the CH2D constructs to form dimers in solution was assessed using size exclusion chromatography. The construct with the IgG hinge 5 and cysteine at the N-terminus formed a unique dimer in PBS at pH 7.4 (FIG. 15). The next set of constructs tested contained two CH2 domains connected by different string linkers (FIG. 16). FLAG tags were added to the carboxy termini in order to be able to detect the expression of these CH2Ds using FLAG-specific antibodies (FIG. 16, first two constructs). Another set of constructs had the HIS-tag added to the NH₂-terminus, and the FLAG-tag was removed and replaced with a stop codon. The sequences for all of the linkers tested are provided in Table 2. The molecular weights of these constructs were determined, using size exclusion chromatography, to be two times that of the CH2D monomer (FIG. 17), indicating dimer formation. An additional hinge from IgG and cysteines at the N- or C-terminal of a stabilized CH2-m01 were added to generate two additional constructs (FIG. 18). Dimer formation was assessed for these constructs using size-exclusion chromatography (FIG. 19). These constructs formed dimers; however, there were lower yields for these variants, as compared to other constructs (Table 1).

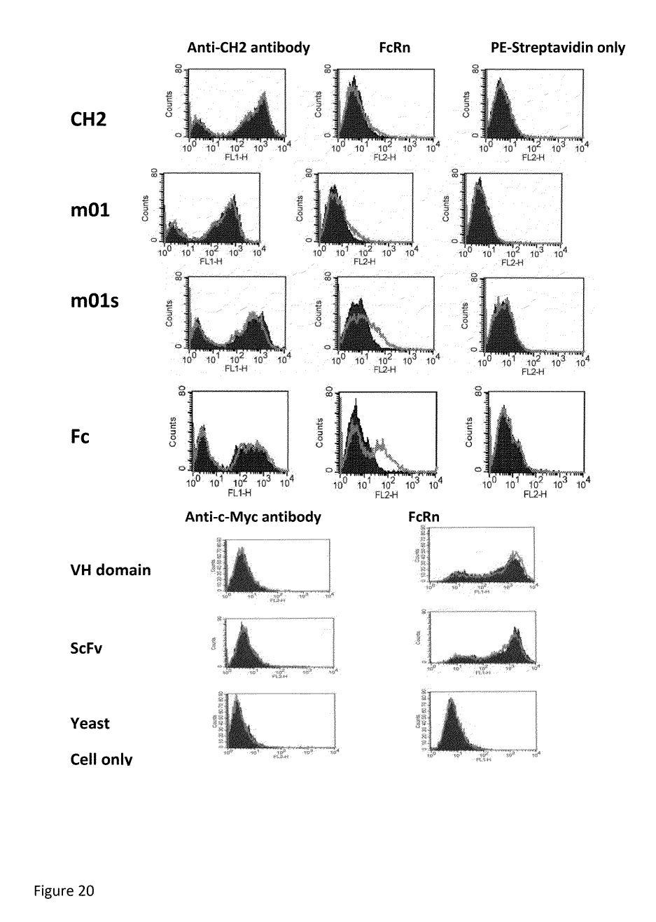

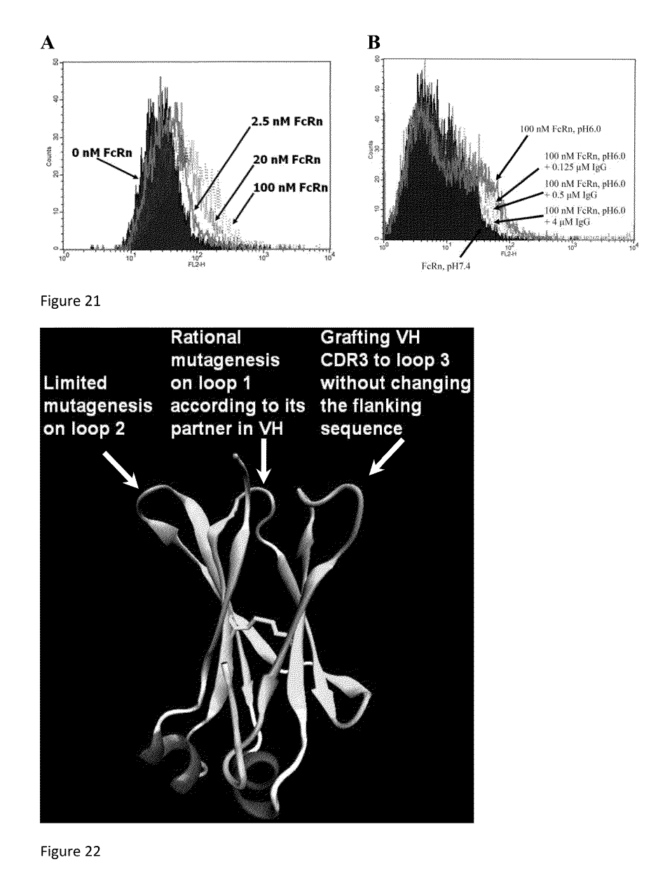

Binding of CH2D to FcRn is known to increase the in vivo half-life of CH2D, so the binding of the various CH2D constructs to FcRn was assessed using a yeast display assay based on FACS analysis (Chao et al. (2006) Nature Protocols 1(2):755-68). CH2, m01, m01s were cloned into the pYD7 vector (Loignon et al. (2008) BMC Biotechnology 8:65), which was developed in Dr. Dimitrov's group and is a modification of pCTCON2 described in Chao et al. (2006) Nature Protocols 1(2):755-68) to promote expression of these proteins on the surface of yeast cells. Fc was also cloned into this vector to serve as a positive control. A VH domain and single chain variable fragment (ScFv) domain were inserted into the same vector to serve as negative controls. A biotin-conjugated single chain FcRn protein was used as a target to test the binding of these domains to FcRn. Expression of all the constructs was confirmed using an anti-CH2D antibody. CH2D was determined to bind to FcRn, although weakly, in a pH-dependent manner; there was increased binding at pH 6.0, as compared to pH 7.4 (FIG. 20, gray vs. black tracing). The extremely stabilized m01s binds more strongly to FcRn than do CH2D or m01 (FIG. 20). This binding was dose-dependent (FIG. 21, left panel) and could be inhibited by IgG (FIG. 21, right panel). These data demonstrate that CH2D, m01 and m01s all bind to FcRn; therefore, they should exhibit high in vivo stability and a longer in vivo half-life, which is necessary for a potential therapeutic.

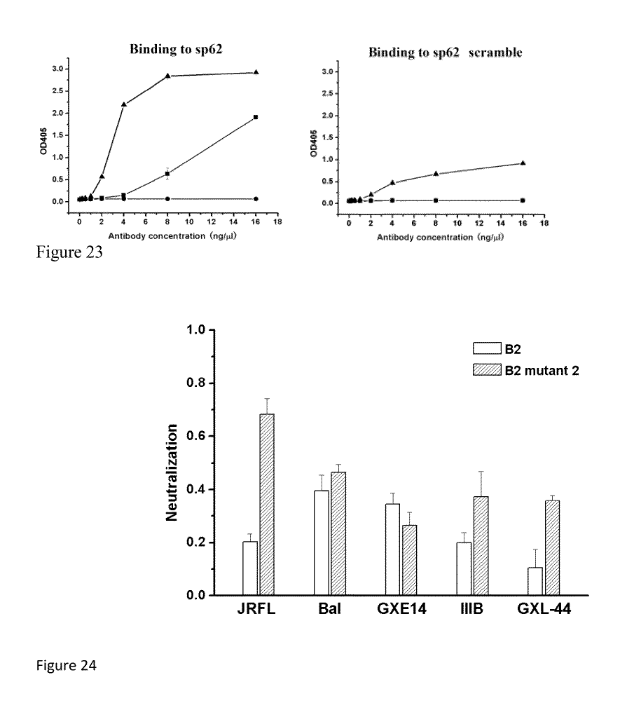

Based on the extremely high stability and FcRn binding of m01s, a CH2D library was generated using m01s as a scaffold. This library was screened against targets of interest in order to identify binders. Specifically, a CH2D library of 10⁸ members was generated using the m01s scaffold (FIG. 22). Mutations were made in all amino acids in loops one and three using only four amino acid residues (Tyrosine, Alanine, Aspartic Acid, or Serine). The length of each loop was fixed, so the diversity of the library was limited. This library design worked well in the past when a wild-type CH2D was used as the scaffold; a highly conserved CD41 epitope was identified (Xiao et al. (2009) BBRC 387(2):387-92). The new m01s-based library was screened against targets of interest. A peptide from the HIV-1 Env membrane proximal external region (MPER) was identified to bind to a member of the library, which was subsequently named B2. A synthetic peptide covering the MPER region, called sp62, was used to assess the binding of B2 to the MPER. An ELISA was performed using previously described methods (Xiao et al. (2009) Biochemical and Biophysical Research Communications 387:387-392). B2 bound to the sp62 protein, and the m01s CH2D did not exhibit binding (FIG. 23, left panel). The binding to a scrambled sp62 was much weaker; this was most likely due to non-specific interactions (FIG. 23, right panel). The positive control for this ELISA assay was the mAb 2F5 (Stiegler et al. (2001) AIDS Res. Hum. Retrovirus 17:1757-1765), which demonstrated strong binding to sp62 and some non-specific binding to the scrambled sp62 construct (FIG. 23).

The antibody response is required to prevent viral infections and may contribute to the resolution of infection. When cells are infected with viral particles, antibodies are produced against many epitopes of the viral proteins. Antibodies can neutralize the function of viruses by multiple methods. The neutralization activity of the CH2D B2 binder against several HIV strains (FIG. 24) was assessed. After the first round of maturation of B2 by yeast display, we obtained a mutant CH2D, which we termed B2 mutant 2. This mutant showed higher neutralization activity against the different HIV-1 strains than B2 (FIG. 24, gray vs. white bars).

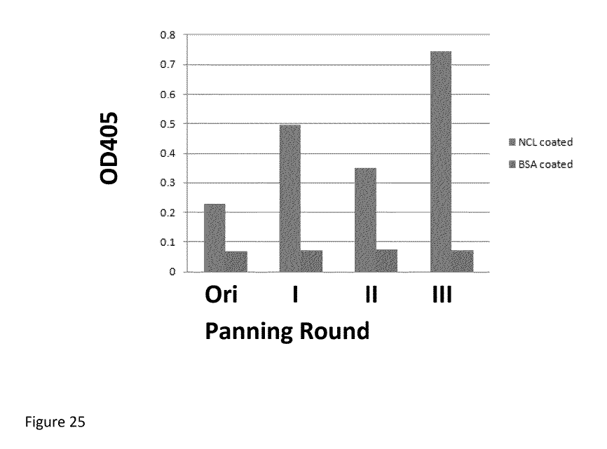

The CH2D library was expressed in yeast surface display and phage display in order to screen for binders against nucleolin using previously described methods (Chao et al. (2006) Nature Protocols 2006; 1(2):755-68). Polyclonal phage ELISA was performed to see whether there was enrichment of antigen-specific phage. There was enrichment of an antigen-specific phage after three rounds of panning (FIG. 25), demonstrating that CH2D binders to nucleolin can be obtained. Screening this library identified additional binders that bound to the HIV proteins, MPER of gp41 and sCD4-gp120, and to sCD4-Balgp120.

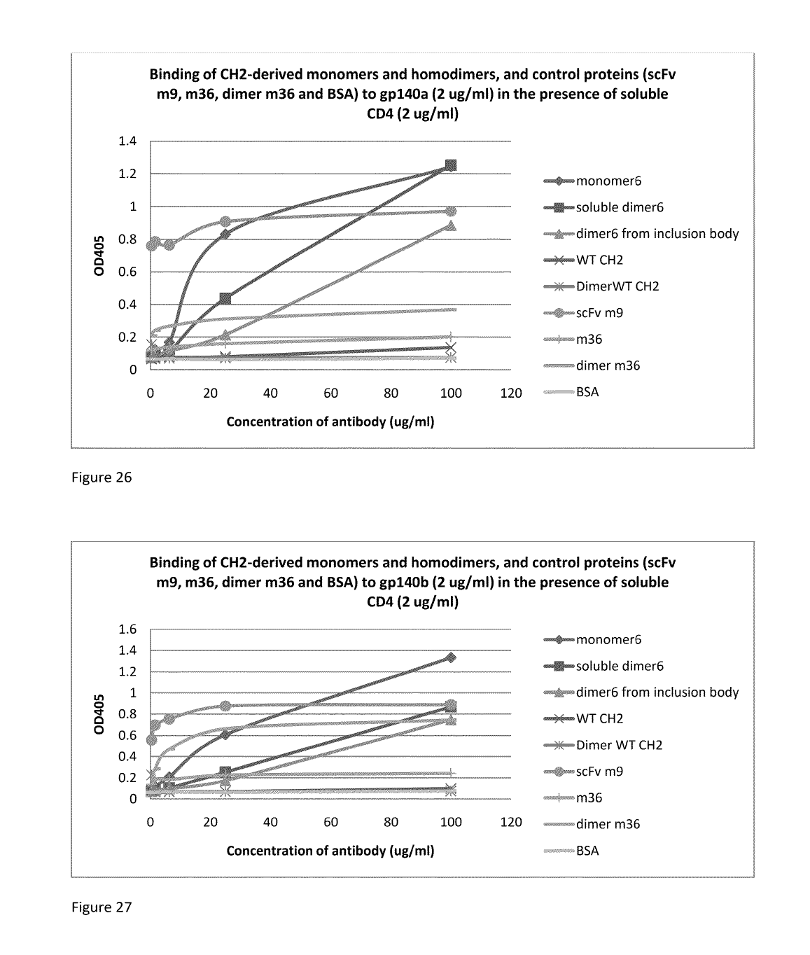

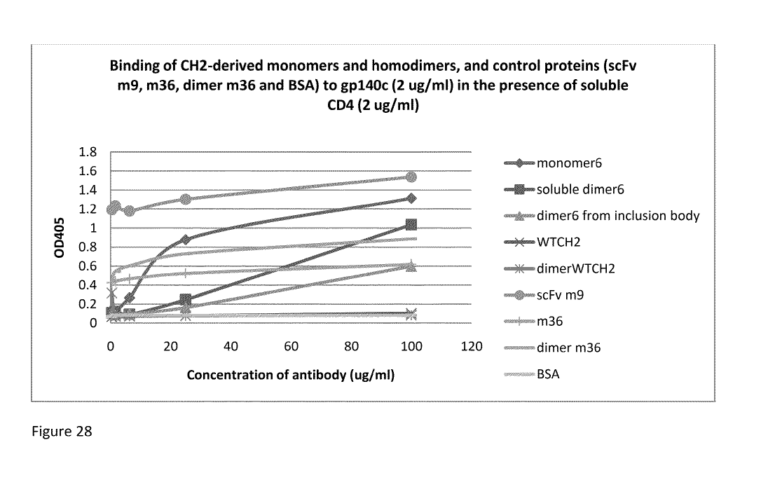

The binding of CH2D monomers and hetero- and homo-dimers to specific targets was assessed and compared. The CH2D binder against CD4-Balgp120 was used to assess homodimerization of CH2D scaffold proteins; this binder is called monomer 6. The homodimer of monomer 6 linked by IgG1hinge12-SSESKYGPPAGG is called dimer 6. Dimer 6 is obtained in soluble form and from the inclusion body of the E. coli microorganisms in which it is expressed. The binding of monomer 6 and dimer 6 to gp140 was compared using ELISA. The wells were coated with antigen (gp140). There were three different gp140s tested for binding to the CH2D monomers and dimers: (1) CH12 gp140 (gp140a); (2) consensus gp140 (gp140b); (3) and SC gp140 (gp140c). The samples were blocked with 5% milk for 1 hr at 37° C. The wells were washed four times with phosphate buffer saline containing Tween 20. CH2D derived monomers or dimers or various controls, in the presence of 2 ug/ml of soluble CD4 (sCD4), were added to the wells at 20 ug/ml, 40 ug/ml, 60 ug/ml, 80 ug/ml, and 100 ug/ml. These solutions were prepared in 1% MPBS. The samples were incubated at 37° C. for 2 hr. The samples were then washed in PBST four times. Secondary antibodies (HRP-anti-Flag tag antibody) were added to the wells at 37° C. for 1 hr. The samples were washed in PBST four times. The chromagen ABTS was added to the wells for 8 minutes at room temperature, and the OD405 absorbance was recorded. The monomer 6 binds to gp140a better than the soluble dimer 6 (FIG. 26, Table 3). The dimer 6 from the inclusion body also exhibits binding to gp140a, but this is lower than that of either monomer 6 or soluble dimer 6. The VH-based engineered antibody domain m36 monomer is described in Chen et al. (PNAS (2008) 105(44): 17121-17126). The m36 monomer also binds to gp140a; however, it has lower affinity than dimer m36 (FIG. 26, Table 3). m36 is an engineered antibody domain based on VH, and monomer 6 is based on CH2 so they have very different sequences which are published, thus the monomers and the dimers significantly differ in their sequence but have overlapping epitopes and likely have 3D structure following the Ig fold. The wild-type monomer and dimer CH2Ds do not bind to gp140a. BSA is a negative control, and scFv m9 is a positive control. Binding to gp140b was similar to the binding observed for gp140a, with the exception that dimer m36 binds more effectively to gp140b than the monomer m36 (FIG. 27, Table 4). These data indicate that there is differential binding of monomers and CH2D homodimers to target molecules. For yet another gp140 (gp140c), monomer 6 demonstrated greater binding than soluble dimer 6 (FIG. 28, Table 5). The dimer 6 refolded from inclusion bodies demonstrated binding, but it was much less than monomer 6 and soluble dimer 6 (FIG. 28). The dimer m36 exhibited better binding to gp140c than the monomer m36 (FIG. 28).

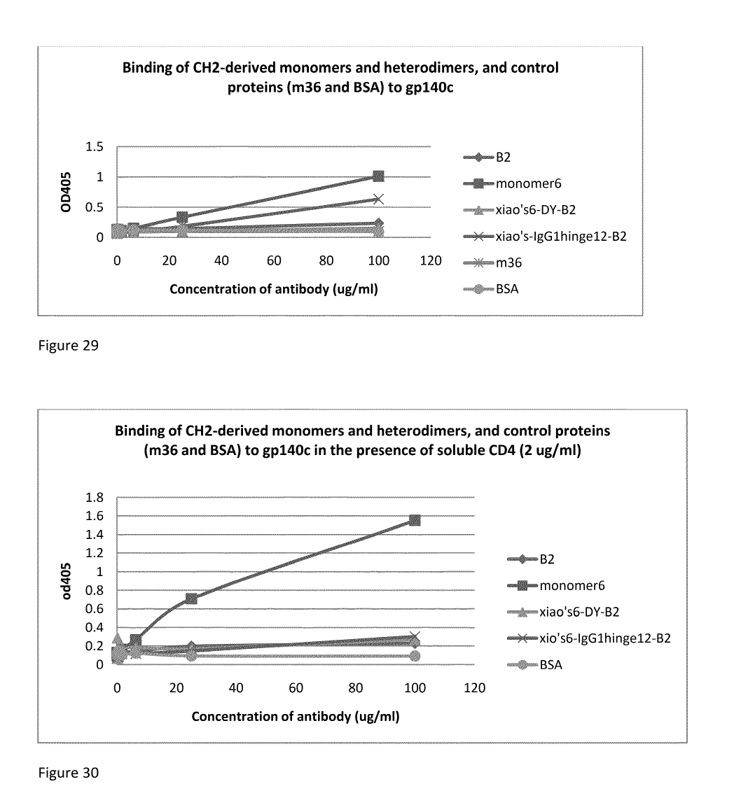

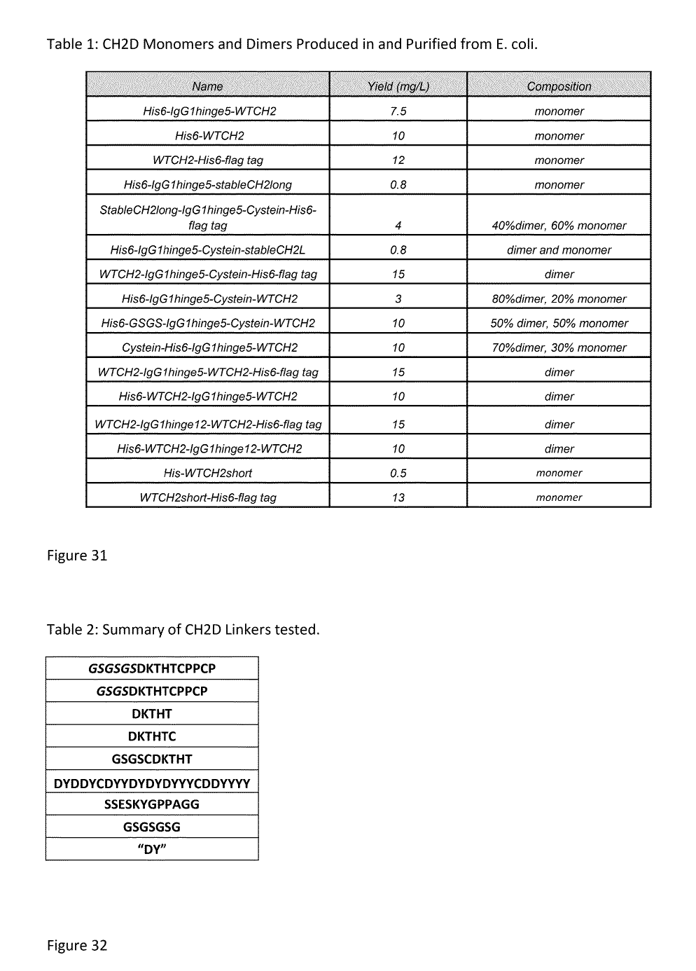

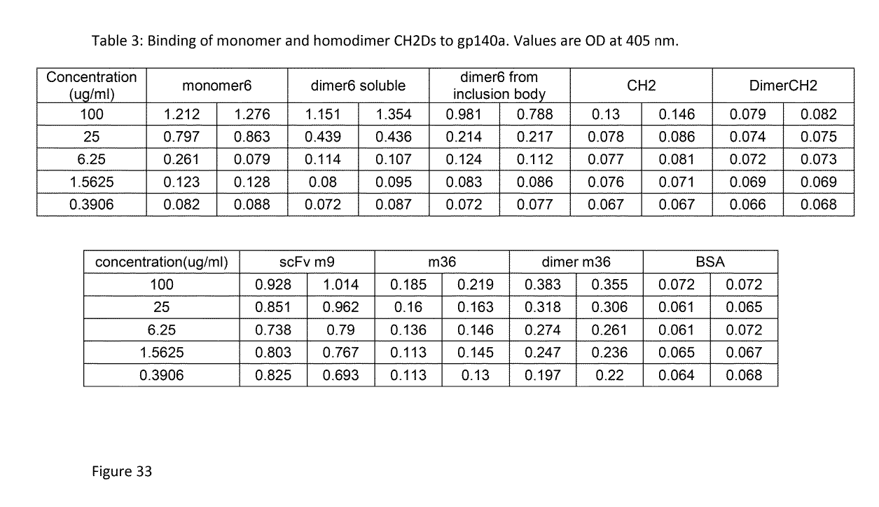

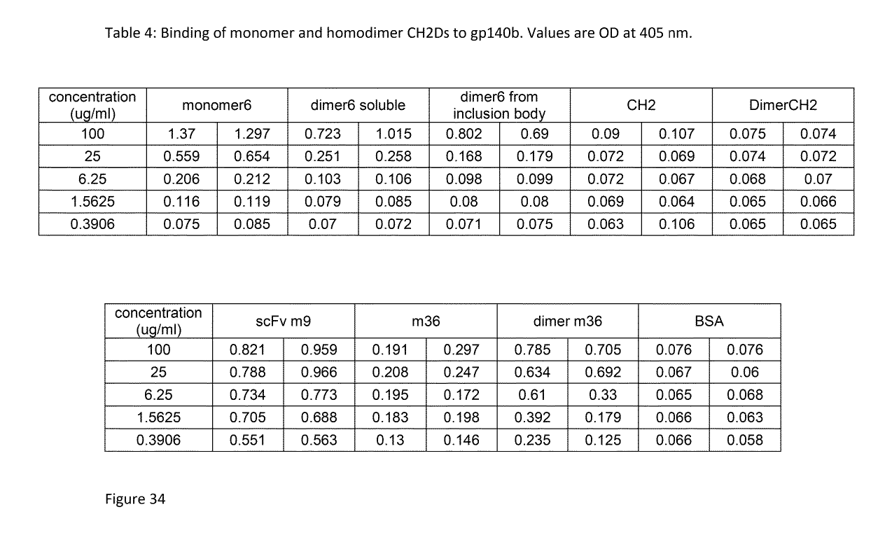

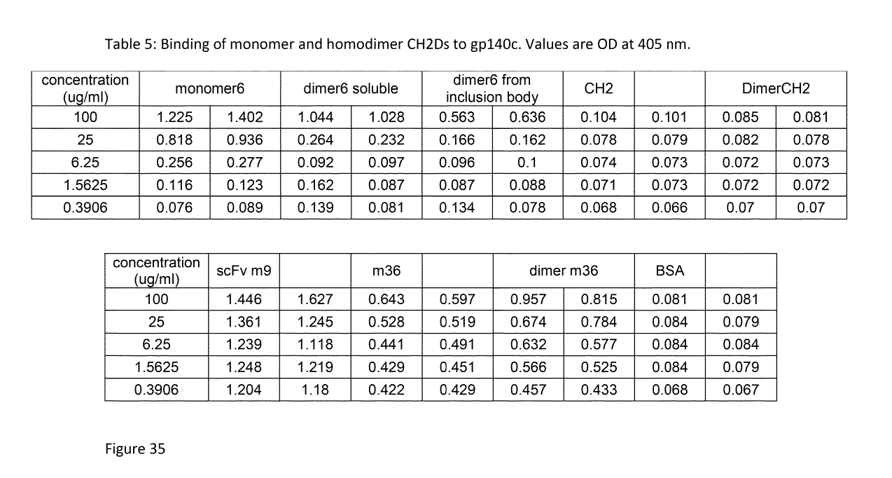

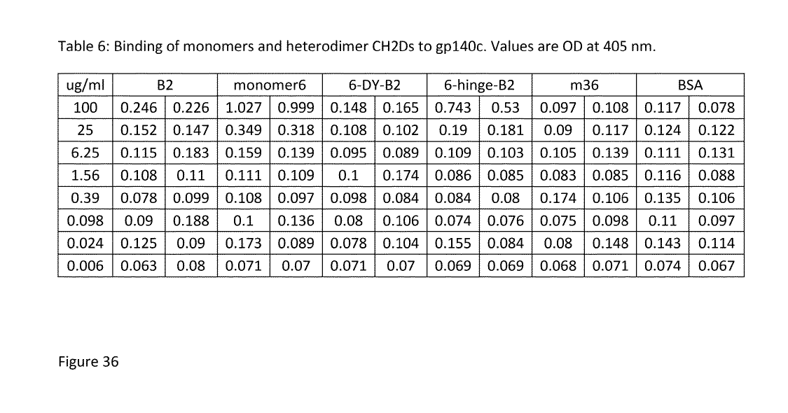

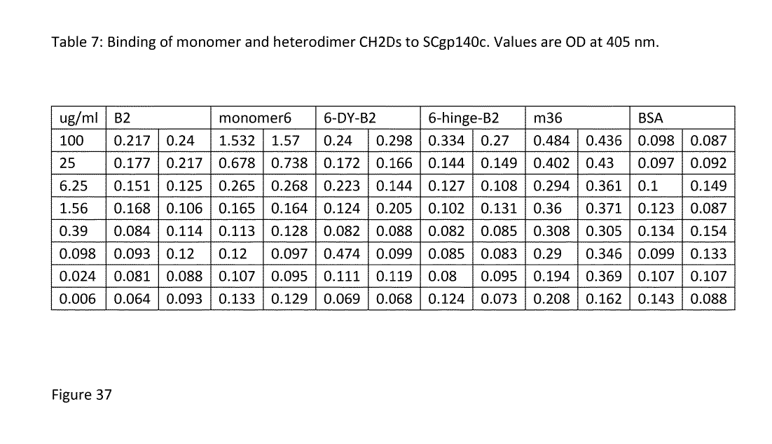

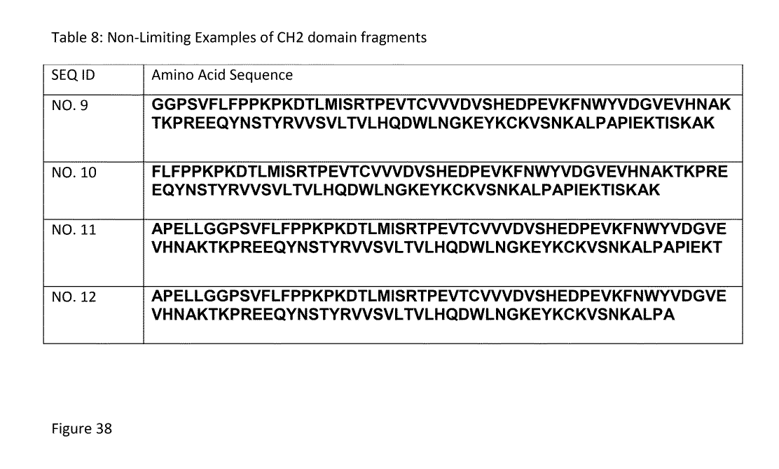

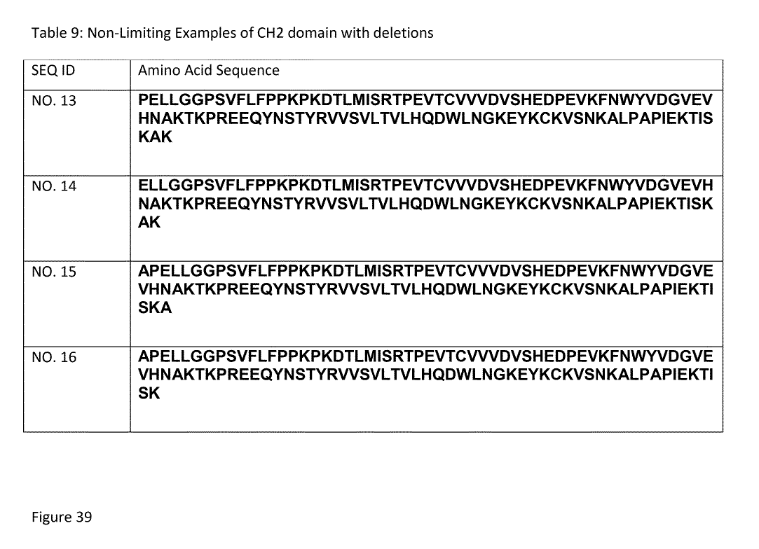

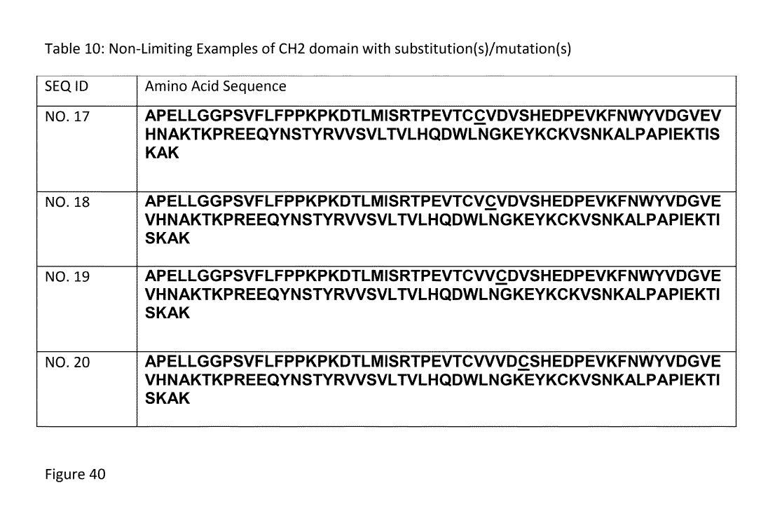

The binder against CD4-Balgp120 (monomer 6) and the binder against SP62 (B2) were used to assess the effect of heterodimerization of CH2D scaffold proteins. Monomer 6 exhibited the greatest binding affinity for the gp140c (FIG. 29, Table 5). The monomer 6-IgG1hinge12-B2 heterodimer exhibited binding to gp140c. The monomer 6-DY-B2 construct did not exhibit high binding affinity to gp140c. The monomer m36 does not bind to gp140c in the absence of sCD4 (FIG. 28 vs. FIG. 29). There is less binding of monomer 6-IgG1hinge12-B2 heterodimer in the presence of sCD4 (FIG. 29 vs. FIG. 30). These results suggest that CH2D monomers, homodimers and heterodimers can bind to various gp140s, although the binding is weak. Further in vitro maturation could increase their binding affinity.

All patent and patent applications mentioned in this application, including the following the disclosures of the following U.S. patents, are incorporated in their entirety by reference herein to the extent that they are consistent with the spirit and claims of the present application: U.S. Patent Application No. 2007/0178082; U.S. Patent Application No. 2007/0135620.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the invention.

What is claimed is:

1. A recombinant CH2 domain (CH2D) multimer comprising a first immunoglobulin CH2 domain linked to a second immunoglobulin CH2 domain, either or both CH2 domains are stabilized and modified compared to a wild-type CH2 domain, at least one CH2 domain comprises at least one structural loop modified to an antigen-binding loop.

2. (canceled)

3. The CH2D multimer of claim 1, wherein the first immunoglobulin CH2 domain and a second immunoglobulin CH2 domain are linked via a linker.

4. The CH2D multimer of claim 3, wherein the linker comprises a peptide between about 5 to 20 amino acids in length.

5. The CH2D multimer of claim 3, wherein the linker comprises at least one multimerizing domain.

6. The CH2D multimer of claim 3, wherein the linker is a hinge component.

7-12. (canceled)

13. The CH2D multimer of claim 1 further comprising a third immunoglobulin CH2 domain.

14. The CH2D multimer of claim 13 further comprising a fourth immunoglobulin CH2 domain.

15-16. (canceled)

17. The CH2D multimer of claim 1, wherein an N-terminus of the first immunoglobulin CH2 domain is linked to a C-terminus of the second immunoglobulin CH2 domain.

18. The CH2D multimer of claim 1, wherein an N-terminus of the second immunoglobulin CH2 domain is linked to a C-terminus of the first immunoglobulin CH2 domain.

19. The CH2D multimer of claim 1, wherein a C-terminus of the first immunoglobulin CH2 domain is linked to a C-terminus of the second immunoglobulin CH2 domain.

20. The CH2D multimer of claim 1, wherein an N-terminus of the first immunoglobulin CH2 domain is linked to an N-terminus of the second immunoglobulin CH2 domain.

21. (canceled)

22. The CH2D multimer of claim 1 wherein the at least one structural loops modified to an antigen-binding loop are designed by rational design, obtained by random mutation, or selected from a diverse library of randomly designed loops.

23. The CH2D multimer of claim 1, wherein the at least one structural loops modified to an antigen-binding loop are obtained by replacing a structural loop with an entire or partial CDR or a functional fragment thereof.

24-25. (canceled)

26. The CH2D multimer of claim 1, wherein at least one loop and at least one strand of the first CH2 domain, the second CH2 domain, or both the first CH2 domain and second CH2 domain are modified.

27-31. (canceled)

32. The CH2D multimer of claim 1 comprising at least one functional FcRn binding site that enhances serum half life of the CH2D multimer.

33-37. (canceled)

38. The CH2D multimer of claim 1, wherein the first immunoglobulin CH2 domain and the second or subsequent immunoglobulin CH2 domain are both specific for a first target.

39. The CH2D multimer of claim 1, wherein the first immunoglobulin CH2 domain is specific for a first target and the second or subsequent immunoglobulin CH2 domain is specific for a second or third target, the first target being different from the second or third target.

40-45. (canceled)

46. A method of neutralizing or destroying a target, said method comprising: (a) obtaining a CH2 domain (CH2D) multimer of claim 1, 13, or 14; (b) introducing the CH2D multimer to a target; and (c) the CH2D multimer binding to the target, the binding functions to cause neutralization or destruction of the target.

47. The method of claim 46, wherein the CH2 multimer comprises an agent, the agent functions to neutralize or destroy the first target.

48. The method of claim 47, wherein the agent is a chemical, a peptide, or a toxin.

49-50. (canceled)

51. A method of detecting a disease or a condition, the method comprising:
(a) obtaining a CH2 domain (CH2D) multimer comprising a first immunoglobulin CH2 domain linked to a second immunoglobulin CH2 domain;
(b) introducing the CH2D multimer into a sample;
(c) detecting binding of the CH2D multimer to a target in the sample, the target being associated with the disease or condition, wherein detecting the binding of the CH2D to the target is indicative of the disease or condition.

52. (canceled)

53. A pharmaceutical composition comprising a CH2 domain (CH2D) multimer of claim 1, 13, or 14; and a pharmaceutical carrier.

54-58. (canceled)

59. A pharmaceutical composition comprising one or more CH2 domains (CH2Ds), stabilized CH2Ds, or multimeric CH2Ds wherein the composition comprises a toxin, drug, biologically active protein or immunotoxin linked to at least one CH2D.

60-68. (canceled)


SEQUENCE LISTING (21 sequences)

SEQ ID NO: 1 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 2 - protein, 110 aa, Homo sapiens
APELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAK

SEQ ID NO: 3 - protein, 103 aa, Homo sapiens
PSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAK

SEQ ID NO: 4 - protein, 109 aa, Homo sapiens
HHHHHHPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAK

SEQ ID NO: 5 - protein, 116 aa, Homo sapiens
HHHHHHAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 6 - protein, 126 aa, Homo sapiens
HHHHHHGSGSCDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 7 - protein, 231 aa, Homo sapiens
HHHHHHAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKDKTHTAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 8 - protein, 116 aa, Homo sapiens
HHHHHHAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAK

SEQ ID NO: 9 - protein, 231 aa, Homo sapiens
HHHHHHAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAKDKTHTAPELLGGPSVFCFPPKPKDTLMISRTPEVTCVVVDVSHEDPEVKFNWYVDGVEVHNAKTKPREEQYNSTYRVVSVLTVLHQDWLNGKEYKCKVSNKALPAPIECTISKAK

SEQ ID NO: 10 - protein, 100 aa, Homo sapiens
FLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 11 - protein, 105 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKT

SEQ ID NO: 12 - protein, 100 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPA

SEQ ID NO: 13 - protein, 109 aa, Homo sapiens
PELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 14 - protein, 108 aa, Homo sapiens
ELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 15 - protein, 109 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKA

SEQ ID NO: 16 - protein, 108 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISK

SEQ ID NO: 17 - protein, 109 aa, Homo sapiens
APELLGGPSVFLFPPKPKDTLMISRTPEVTCCVDVSHEDPEVKFNWYVDGVEVHNAKTKPREEQYNSTYRVVSVLTVLHQDWLNGKEYKCKVSNKALPAPIEKTISKAK

SEQ ID NO: 18 - protein, 110 aa, Homo sapiens
APELLGGPSVFLFPPKPKDTLMISRTPEVTCVCVDVSHEDPEVKFNWYVDGVEVHNAKTKPREEQYNSTYRVVSVLTVLHQDWLNGKEYKCKVSNKALPAPIEKTISKAK

SEQ ID NO: 19 - protein, 110 aa, Homo sapiens
APELLGGPSVFLFPPKPKDTLMISRTPEVTCVVCDVSHEDPEVKFNWYVDGVEVHNAKTKPREEQYNSTYRVVSVLTVLHQDWLNGKEYKCKVSNKALPAPIEKTISKAK

SEQ ID NO: 20 - protein, 110 aa, Homo sapiens
APELLGGPSVFLFPPKPKDTLMISRTPEVTCVVVDCSHEDPEVKFNWYVDGVEVHNAKTKPREEQYNSTYRVVSVLTVLHQDWLNGKEYKCKVSNKALPAPIEKTISKAK

SEQ ID NO: 21 - protein, 105 aa, Homo sapiens
GGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK